(19) United States
(12) Patent Application Publication
(10) Pub. No.: US 2005/0158408 A1
(43) Pub. Date: Jul. 21, 2005

(54) Dried forms of aqueous solubilized bile acid dosage formulation: preparation and uses thereof

(76) Inventor: Seo Hong Yoo, Wyckoff, NJ (US)

Correspondence Address: BAKER & BOTTS, 30 ROCKEFELLER PLAZA, NEW YORK, NY, 10112, US

(21) Appl. No.: 10/996,945

(22) Filed: Nov. 24, 2004

Related U.S. Application Data
(63) Continuation-in-part of application No. 09/778,154, filed on Feb. 5, 2001.
Application No. 09/778,154 is a continuation-in-part of application No. 09/357,549, filed on Jul. 20, 1999, now Pat. No. 6,251,428.
(60) Provisional application No. 60/094,069, filed on Jul. 24, 1998.

(51) Int. Cl. A61K 31/56; A61K 35/78; A61K 31/198

(52) U.S. Cl. 424/728; 514/170; 514/561

(57) ABSTRACT

Compositions for pharmaceutical and other uses comprising clear aqueous solutions of bile acids which do not form any detectable precipitates over selected ranges of pH values of the aqueous solution and methods of making such solutions are disclosed. Compositions of the disclosure may comprise water; a bile acid in the form of a bile acid, bile acid salt, or a bile acid conjugated with an amine by an amide linkage; and either or both an aqueous soluble starch conversion product and an aqueous soluble non-starch polysaccharide. The composition remains in solution without forming a precipitate over a range of all pH values obtainable in an aqueous system. The composition, according to some embodiments, may further contain a pharmaceutical compound in a pharmaceutically effective amount. The disclosure further provides dried forms of primary aqueous solubilized bile acid formulations and methods of preparing such dried forms.

This application is a continuation in part of application Ser. No. 09/778,154 filed Feb. 5, 2001 which is a continuation in part of application Ser. No. 09/357,549 filed Jul. 2, 1999 which claims the benefit of provisional application No. 60/094,069, filed Jul. 24, 1998, all of which are incorporated herein by reference.

### BACKGROUND OF THE INVENTION

Bile acids salts, which are organic acids derived from cholesterol are natural ionic detergents that play a pivotal role in the absorption, transport, and secretion of lipids. The term, primary bile acid refers to those synthesized de novo by the liver. In humans, the primary bile acids include cholic acid (3α, 7α, 12α-trihydroxy-5 β-cholanic acid) (“CA”) and chenodeoxycholic acid (3α, 7α-dihydroxy-5 β-cholanic acid) (“CDCA”). Dehydroxylation of these bile acids by intestinal bacteria produces the more hydrophobic secondary bile acids, deoxycholic acid (3α, 12α-dihydroxy-5β-cholanic acid) (“DCA”) and lithocholic acid (3α-hydroxy-5 β-cholanic acid) (“LCA”). These four bile acids CA, CDCA, DCA, and LCA, generally constitute greater than 99 percent of the bile salt pool in humans. Secondary bile acids that have been further metabolized by the liver are sometimes denoted as tertiary bile acids.

Keto-bile acids are produced secondarily in humans as a consequence of oxidation of bile acid hydroxyl groups, particularly the 7-hydroxyl group, by colonic bacteria. However, keto-bile acids are rapidly reduced by the liver to the corresponding α or β-hydroxy bile acids. For example, the corresponding keto bile acid of a CDCA is 7-keto lithocholic acid and one of its reduction products with the corresponding β-hydroxy bile acid is ursodeoxycholic acid (3α-7β-dihydroxy-5 β-cholanic acid) (“UDCA”), a tertiary bile acid.

Bile acids containing a 6β-hydroxyl group, which are found in rats and mice, are known as muricholic acid; 6α-hydroxy bile acids produced by swine are termed hyocholic acid and hyodeoxycholic acids. 23-hydroxy bile acids of aquatic mammals are known as phocecholic and phocedeoxycholic acids.

Typically, more than 99 percent of naturally occurring bile salts secreted into human bile are conjugated. Conjugates are bile acids in which a second organic substituent (e.g. glycine, taurine, glucuronate, sulfate or, rarely, other substituents) is attached to the side chain carboxylic acid or to one of the ring hydroxyl groups via an ester, ether, or amide linkage. Therefore, the ionization properties of conjugated bile acids with glycine or taurine are determined by the acidity of the glycine or taurine substituent.

Free, unconjugated, bile acid monomers have pKₐ values of approximately 5.0. However, pKₐ values of glycine conjugated bile acids are on average 3.9, and the pKₐ of taurine conjugate bile acids are less than 1.0. The effect of conjugation, therefore, is to reduce the pKₐ of a bile acid so that a large fraction is ionized at any given pH. Since the ionized salt form is more water soluble than the protonated acid form, conjugation enhances solubility at a low pH. Free bile acid salts precipitate from aqueous solution at pH 6.5 to 7. In contrast, precipitation of glycine conjugated bile acid occurs only at pH of less than 5. Taurine conjugated bile acids remain in aqueous solution under very strongly acidic conditions (lower than pH 1). However, in the gastric pH range, certain bile acids such as UDCA and CDCA are no longer soluble.

Conjugation of the side chain of a bile acid with glycine or taurine has little influence on the hydrophobic activity of fully ionized bile salts. More hydrophobic bile salts exhibit greater solubilizing capacity for phospholipid and cholesterol and are consequently better detergents. More hydrophobic bile salts are also more injurious to various membranes, both in vivo and in vitro.

Natural bile salt pools invariably contain multiple bile acid salts. Mixtures of two or more bile salts of differing hydrophobic activity may behave as a single bile salt of an intermediate hydrophobic activity. As a result, detergent properties and the toxicity of mixtures of two bile acids of differing hydrophobic activity often are intermediate between the individual components.

Bile acids have a variety of properties. For example, UDCA may be a useful immuno-modulating agent. It may also inhibit induction of nitric oxide synthase (NOS) in human intestinal epithelial cells and in vivo. Bile acids may act as pepsin inhibitors, with UDCA being the most potent. In addition, bile acids may have membrane stabilizing properties. UDCA is a prototype of a novel and selective glucocorticoid receptor (GR) modifier and represses NF-kB without induction of transactivation function of the GR. In addition, UDCA plays a unique role in modulating the apoptotic threshold to a variety of agents acting through different apoptotic pathways in both hepatic and non-hepatic cells. Finally, UDCA has specific antioxidant properties. The OH free radical scavenging efficiency of UDCA appears remarkable in that its rate constant for reaction with this radical species is about ten-fold higher than that of the well known pharmacological scavenger mannitol and of the physiological scavengers glucose or histidine. This scavenging activity may give rise to the ability of UDCA to inhibit deoxycholic acid-induced apoptosis by modulating mitochondrial transmembrane potential and reactive oxygen species production.

Bile flow is generated by the flux of bile salts passing through the liver. Ursodeoxycholic acid may promote bile flow by inducing hepatocytes to release ATP into bile, which then stimulates fluid and electrolyte secretion by bile-duct epithelia downstream via changes in cytosolic Ca++. Bile salts in the enterohepatic circulation are thought to regulate bile acid synthesis by suppressing or derepressing the activity of cholesterol 7-hydroxylase, which is the rate-limiting enzyme in the bile acid biosynthesis pathway. Bile formation represents an important pathway for solubilization and excretion of organic compounds, such as bilirubin, endogenous metabolites, such as amphipathic derivatives of steroid hormones, and a variety of drugs and other xenobiotics.

Bile acids may play a role in the regulation of hepatic lipoprotein receptors (apo B.E.) and consequently may modulate the rate of uptake of lipoprotein cholesterol by the liver. Secretion of bile salts into bile, on the other hand, is coupled with the secretion of two other biliary lipids, phosphatidylcholine (lecithin) and cholesterol. Coupling bile salt output with the lecithin and cholesterol output provides a major pathway for the elimination of hepatic cholesterol. Bile acids may also be a factor in the regulation of cholesterol synthesis by acting directly on the hydroxymethylglutaryl-coenzyme A (HMG-CoA) reductase or indirectly by modulating the cholesterol absorption in the intestine. Bile salts, along with lecithin, solubilize cholesterol in bile in the form of mixed micelles and vesicles. In the intestines, bile salts in the form of mixed micelles participate in the intraluminal solubilization, transport, and absorption of cholesterol, fat-soluble vitamins, and other lipids. Bile salts may be involved in the transport of calcium and iron from the intestinal lumen to the brush border.

UDCA, a major component of bear bile, has been used as a major pharmaceutical agent for the treatment of and protection against many types of liver disease. Its medicinal uses include the dissolution of radiolucent gall stones, the treatment of biliary dyspepsia, primarily biliary cirrhosis, primary sclerosing cholangitis, chronic active hepatitis and hepatitis C. High levels of bile acids remarkably inhibit the proliferation of hepatitis C virus.

The hydrophilic nature of UDCA may confer cytoprotection in necroinflammatory diseases of the liver. UDCA also significantly improves transaminases and cholestatic enzymatic indices of liver injury in chronic hepatitis and alleviates alcoholic fatty liver. Bile salt deficiency, and consequently reduced cholesterol solubility in bile, may play a role in the pathogenesis of cholesterol gallstones.

Bile acids may also have significant therapeutic value in treating a number of other conditions including those that affect the heart and the gastrointestinal tract. For example, UDCA has a vasodilative effect on the systemic vascular bed, but altered neither pulmonary vascular function nor cardiac functions. Regarding the gastrointestinal tract, bile acids substantially inhibit the growth of H. pylori.

In spite of the potentially valuable medical uses of bile acids as therapeutically active agents and as carriers and/or adjuvants, commercial use of bile acids is limited to pharmaceutical formulations with a solid form of bile acid which are in tablet, capsule and suspension. This is due to the insolubility of bile acids in aqueous media at pH from approximately 1 to 8. This is also due to bile's extremely bitter taste and equally bitter after-taste which lasts several hours. The few aqueous dosage forms that are available are unstable, and have very limited uses because of pH control and maintenance problems. Moreover, some commercial pharmaceutical dosage forms of bile acids have been shown to have scant bioavailability. This is even true of solid bile acid forms.

Therefore, a need has arisen for an liquid and solid bile acid formulation that are (liquids) or form (solids) clear, aqueous solutions.

### SUMMARY OF THE INVENTION

Bile acid compositions may be advantageously stored or administered in a dry or solid form. Thus, the present invention relates to dry or solid preparations of bile acids that form clear or particulate-free solutions upon exposure to water. Dry or solid forms of the invention may be prepared from clear or particulate-free solutions of bile acids (“parent solutions”). The present invention also relates to methods for preparing and/or solubilizing such dry or solid forms. Advantages of these formulations include improved bioavailability, plasma bioavailability, and absorbability of a bile acid. Additional advantages of formulations of the invention include improved bioavailability, plasma bioavailability and absorbability of one or more pharmaceutical compounds.

In some preferred embodiments, a dry or solid preparation of the invention exposed to water results in a solution comprising (1) a bile acid, its derivative, its salt, or its conjugate with an amine, (2) water, and (3) a sufficient quantity of an aqueous soluble starch conversion product such that the bile acid and the starch conversion product remain in solution at any pH within a selected pH range. According to some preferred embodiments, a dry or solid preparation of the invention exposed to water results in a solution comprising (1) a bile acid, its derivative, its salt, or its conjugate with an amine, (2) water, and (3) a sufficient quantity of an aqueous soluble non-starch polysaccharide and an aqueous soluble starch conversion product such that the bile acid and the polysaccharide remain in solution at any pH within a selected pH range.

Dry or solid forms of the invention exposed to water may result in solutions further comprising resistant maltodextrin, an aqueous soluble ginseng extract, a pharmaceutical compound in a pharmaceutically appropriate amount, an aqueous soluble bismuth compound, or combinations thereof. Where the solution comprises one or more such materials, the solution composition may be adjusted to ensure that these materials remain in solution.

Dry or solid preparations of the invention may comprise one or more disintegrants. In some embodiments of the invention, solution formulations of bile acid compositions comprise a disintegrants in order to facilitate breakup or disintegration of its dried forms after administration. The pH of solutions of the invention may be adjusted with high throughput sonication by acid. In some non-limiting embodiments of the invention, high throughput sonication accelerates solubilization of dry or solid bile acid preparations.

Dry or solid forms of the invention are prepared from clear or particulate-free parent solutions. In some embodiments of the invention, dried forms derived from the solution formulations of bile acid compositions may be granulated by the method of granulation evolved from the fluid-bed drying technology. In this system a soluble fiber solution (granulating solution) is sprayed into or onto the suspended dried forms, which then would be dried rapidly in the suspending air.

In some embodiments of the invention, a composition is provided which comprises (1) a bile acid, its derivative, its salt, or its conjugate with an amine, (2) water, and (3) a sufficient quantity of carbohydrate such that the bile acid component and the carbohydrate remain in solution at any pH within a selected pH range, wherein the carbohydrate is a combination of an aqueous soluble starch conversion product and an aqueous soluble non-starch polysaccharide. In embodiments containing both soluble non-starch polysaccharide and high molecular weight starch conversion product, the amounts of each are such that when combined together in the composition they are sufficient to allow the bile acid component, the high molecular weight starch conversion product, the soluble non-starch polysaccharide and the pharmaceutical compound, if any, to remain in solution at any pH within a selected pH range.

In some embodiments of the invention, a combination therapy composition is provided which may increase the intensity of a response to or efficacy of a pharmaceutical. Such a composition may permit administration of lower dosages of a pharmaceutical compound, attack a disease complex at different points, affect elimination and/or alter absorption of a pharmaceutical compound. Such a composition may lead to or contribute to a reduction in toxicity and/or side effects of a pharmaceutical.

In some embodiments, bile solutions of the invention are dried. The invention further relates to dried forms derived from the solution formulations of bile acid compositions by lyophilization, evaporation, or any other means of dehydration known in the art. The solutions may be partially dried to produce a semi-solid forms. The solutions may be thoroughly dried to form a solid, powder and granule. Dried forms of the aqueous solutions may be substantially free of water. Dried forms may be dried by fluid process, tray process, spray process, and freezing process. Dried forms may be administered directly, as solid dosage forms or combined with water prior to administration.

The invention further relates to a method of treating or preventing a human or animal disease comprising administration of a composition of the invention.

### BRIEF DESCRIPTION OF THE DRAWINGS

FIG. 1: Graph of blood serum—concentration of UDCA (squares) and GUDCA (triangles) versus time following administration of dosage formulations according to Examples II and VI and Table 4.

FIG. 2: Graph of blood serum concentration of UDCA versus time following administration of dosage formulations of the bile acid according to Examples III and VI and Table 4.

FIG. 3: Diagram of the mean (n=5) for group I for pharmacokinetic parameters of UDCA in human after an oral administration of liquid formulation of UDCA prepared according to Example IX without bismuth.

FIG. 4: Diagram of the mean (n=5) for group II for pharmacokinetic parameters of UDCA in human after an oral administration of liquid formulation of UDCA prepared according to Example IX.

FIG. 5A. Transmission electron micrograph of H. pylori cultured from Columbia medium.

FIG. 5B. Transmission electron micrograph of H. pylori 48 hrs after being treated with UDCA & bismuth citrate prepared according to Example IX.

FIG. 5C. Transmission electron micrograph of H. pylori 72 hrs after being treated with UDCA & bismuth citrate.

FIG. 6: NMR data for UDCA in a liquid formulation dosage form prepared according to Example III without preservatives, flavoring agent, and sweetener.

FIG. 7: HPLC trace of UDCA in a liquid formulation dosage form prepared according to Example III without preservatives, flavoring agent, and sweetener.

FIG. 8: HPLC trace of a UDCA standard.

FIG. 9: H. pylori culture method.

FIG. 10: H. pylori culture method.

FIG. 11: H. pylori culture method.

FIG. 12: Plot of pH vs. transparency of secondary aqueous solubilized bile acid solution dosage formulations prepared according to Example XIX with regard to the redissolution of dried form derived from a primary aqueous solubilized bile acid formulation within 2 minute. The primary solutions, 100 mL each, comprised 200 mg UDCA and (A) 4 g, (B) 5 g, (C) 6 g, (D) 7 g, (E) 8 g or (F) 9 g of maltodextrin (DE=15).

### DETAILED DESCRIPTION OF THE INVENTION

The present invention relates to an aqueous solution comprising (i) one or more bile acids selected from the group consisting of a soluble bile acid, an aqueous soluble bile acid derivative, a bile acid salt, or a bile acid conjugated with an amine, (collectively “bile acid”), (ii) water, and (iii) one or more aqueous soluble starch conversion products or aqueous soluble non-starch polysaccharide in an amount sufficient to produce a solution which does not form a precipitate at any pH within a desired pH range.

The composition may contain a bile acid or its salt which itself has pharmaceutical effectiveness. Formulations of the invention may act as a carrier, an adjuvant or enhancer for the delivery of a pharmaceutical material which remains dissolved in the composition of the invention across the desired pH range. In some embodiments of the invention, a non-bile acid pharmaceutical is used though not necessarily in solution.

It is an advantage of this invention that the bile acid and the carbohydrate remain in solution without precipitation at any pH from acidic to alkaline. These aqueous solution systems of bile acid are absolutely free of precipitate or particles. A further advantage of this invention is that the aqueous solution systems demonstrate no changes in physical appearance such as changes in clarity, color or odor following the addition of strong acids or alkali even after several months of observation under accelerated conditions of storage at 50° C.

In some embodiments of the invention, an aqueous solution system of bile acid is administered orally whereupon it moves through the gastrointestinal track without precipitation of bile acids as solids by exposure to acidic gastric juices and into the alkaline environment of the intestine. These formulations demonstrate that intact bile acid solution systems in the gastro-intestinal tract can be effectively and completely absorbed.

According to the invention, bile acid solubility (e.g. precipitation and changes in physical appearance) is unaffected by whether a carboxylic acid side chain of certain bile acids can be protonated (non-ionized) or ionized or is a simple carboxylic acid. The ionization state of a bile acid carboxylic acid side chain greatly affects the ability of micelle formation by the bile acid in these aqueous solution systems. In some embodiments of the invention, that ionization state is manipulated by adjusting the pH to control the micelle formation of bile acids with a drug in order to use these aqueous solution systems as a therapeutically active agent, as an adjuvant of a drug, as a carrier of drug or as an enhancer of drug solubility. These aqueous solution systems may be prepared for oral consumption, enemas, mouthwashes, gargles, nasal preparations, otic preparations, injections, douches, topical skin preparations, other topical preparations, and cosmetic preparations which have a desired pH without the disadvantage of precipitation or deterioration in physical appearance after long periods of time.

Soluble bile acids are any type of aqueous soluble bile acids. A bile acid salt is any aqueous soluble salt of a bile acid. The soluble bile acid derivatives of this invention are those derivatives which are as soluble as or more soluble in aqueous solution than is the corresponding underivatized bile acid. Bile acid derivatives include, but are not limited to derivatives formed at the hydroxyl and carboxylic acid groups of the bile acid with other functional groups including but not limited to halogens and amino groups. Aqueous dissolved salts of bile acids may be formed by the reaction of bile acids described above and an amine including but not limited to aliphatic free amines such as trientine, diethylene triamine, tetraethylene pentamine, and basic amino acids such as arginine, lysine, omithine, and amino sugars such as D-glucamine, N-alkylglucamines, and quaternary ammonium derivatives such as choline, heterocyclic amines such as piperazine, N-alkylpiperazine, piperidine, N-alkylpiperidine, morpholine, N-alkylmorphline, pyrrolidine, triethanolamine, and trimethanolamine. According to the invention, aqueous soluble metal salts of bile acids and aqueous soluble O-sulfonated bile acids are also included as soluble bile acid salts.

Bile acids of the invention may be selected from the group consisting of chenodeoxycholic acid, cholic acid, hyodeoxycholic acid, deoxycholic acid, 7-oxolithocholic acid, lithocholic acid, iododeoxycholic acid, iocholic acid, tauroursodeoxycholic acid, taurochenodeoxycholic acid, taurodeoxycholic acid, taurolithocholic acid, glycoursodeoxycholic acid, taurocholic acid, glycocholic acid, and their derivatives at a hydroxyl or carboxylic acid group on the steroid nucleus. In addition, bile acids of the invention may be selected from primary, secondary, and tertiary bile acids.

UDCA is practically insoluble at pH<7. The pKa of UDCA is 5.1, and the solubility of its protonated form is 9 μmol/L. The solubility of UDCA in the solution formulation is about 100 mg/ml which is equivalent to almost 30,000 folds of intact UDCA's solubility. Therefore, the major advantage of the instant invention is that by delivery of solubilized bile acid in solution, it achieves high in vivo levels of bile acids (bile, blood, etc.) far beyond other preparations. Therefore, the therapeutic potential of bile acid may be more fully achieved on the ground of high concentration of proper bile acid in each lesion by systemic supply than previous formulations. The in vivo levels of bile acids attainable with existing formulations in which bile is incompletely and slowly solubilized are lower. Moreover, its low absorption and enterohepatic circulatory action result from non-detection of therapeutically active bile acid such as UDCA in the blood. Since bile acid is completely dissolved in the inventive formulations, higher in vivo levels of bile acid may be achieved, even though lower doses are administered.

In some embodiments of the invention, pluralities of bile acids are used in a single formulation. Mixtures of two or more bile salts of differing hydrophobic activity may behave as a single bile salt of an intermediate hydrophobic activity. As a result, mixtures of two or more bile salts may have the unique physiological advantages such as increased detergent properties and lowered toxicity than the individual bile acids.

Carbohydrates suitable for use in the invention include aqueous soluble starch conversion products and aqueous soluble non-starch polysaccharides. Aqueous soluble starch conversion products may be obtained under various pH conditions from the partial or incomplete hydrolysis of starch. They may also have a Dextrose Equivalent (DE) of from about 4 to about 40. DE is a quantitative measure of the degree of starch polymer hydrolysis. It is a measure of reducing power compared to a dextrose standard of 100. The higher the DE, the greater the extent of starch hydrolysis implies. As the product is further hydrolyzed (higher), the average molecular weight decreases. Non-limiting examples include maltodextrin, dextrin, liquid glucose, corn syrup solid (dried powder of liquid glucose), and soluble starch, preferably maltodextrin or corn syrup solid, most preferably corn syrup solid. For the purpose of this invention, the term “corn syrup” includes both corn syrup and liquid glucose. Aqueous soluble non-starch polysaccharides may be aqueous soluble fiber such as guar gum, pectin, psyllium, oat gum, soybean fiber, oat bran, corn bran, cellulose and wheat bran.

The amount of high molecular weight aqueous soluble starch conversion product used in the invention is at least the amount needed to render the chosen bile acid salt soluble in the concentration desired and in the pH range desired. The approximate minimal quantity of maltodextrin required to prevent the precipitation of bile acids from the aqueous solution formulations of the invention depended on its DE value. In preferred embodiments of the invention, the approximate minimal quantity of maltodextrin which has 15-25 DE value such as Maltrin®M50, Maltrin®M180, Maltrin®M200, Maltrin®M250 (corn syrup solid), liquid glucose, and soluble starch required to prevent the precipitation of bile acids from the aqueous solution formulations of the invention is approximately 30 g for CDCA, approximately 5 g for UDCA, approximately 12 g for 7-ketolithocholic acid (KLCA), approximately 10 g for cholic acid, approximately 50 g for deoxycholic acid, approximately 3.5 g for hyodeoxycholic acid for every 0.2 g of bile acid. In preferred embodiments of the invention, the approximate minimal quantity of a maltodextrin (DE 5-10), such as Maltrin®M040, Maltrin®M100 is approximately 18 g for CDCA, approximately 3 g for UDCA, approximately 7g for 7-ketolithocholic acid, approximately 6 g for cholic acid, approximately 30 g for deoxycholic acid, approximately 2.1 g for hyodeoxycholic acid for every 0.2 g of bile acid.

Digestion resistant maltodextrin is an aqueous soluble dietary fiber. This soluble resistant maltodextrin is produced from corn starch (similar to the process to manufacture conventional maltodextrin) to purposefully convert a portion of the normal alpha-1,4 glucose linkages to random 1,2-, 1,3-, and 1,4-alpha or beta linkages. The human digestive system effectively digests only alpha 1,4-linkages; therefore the other linkages render the molecules resistant to digestion. Thus, other linkages created are not absorbed in the small intestine and passed on to the large intestine. This resistant maltodextrin is partially fermented in the large intestine with the fractions that aren't utilized excreted. This aqueous soluble maltodextrin helps maintain normal, healthy levels of serum cholesterol, blood triglycerides, blood glucose level, intestinal regularity, and intestinal microflora. In some embodiments of the invention, a solution formulation may comprise an aqueous soluble digestion resistant maltodextrin.

In some embodiments of the invention, a formulation may comprise cyclodextrin.

Drugs substances most frequently are administered orally by means of solid dosage forms such as powder, dried granular mass, tablets and capsules. The solid dosage forms can facilitate handling, enhance the physical appearance and improve stability. In many cases, it has been shown that a drug substance's solubility and other physicochemical characteristics influence its physiological availability from a solid dosage form. Dried form contains aqueous soluble bile acid, readily soluble high molecular weight starch conversion product, and disintegrants, which are easy for solubilization. Increased solubility of bile acid leads to the increased rate of dissolution. As a result, the rate of absorption may be increased greatly by the increased rate of dissolution.

Compositions of the invention may further comprise a disintegrant to facilitate breakup or disintegration of a dry or solid form after administration. Disintegrants may be starches such as Veegum HV, methylcellulose, agar, bentonite, natural sponge, cation exchange resins, alginic acid, guar gum, citrus pulp, and carboxymethylcellulose, clays, celluloses, aligns, gums, and cross-linked polymers (crospovidone), cross-linked cellulose (Croscarmelose), and cross-linked starch (sodium starch glycolate). The disintegrating function is due to capillary action rather than swelling. In general, the aqueous soluble disintegrants may be mixed with the active ingredients prior to drying. In case of aqueous insoluble disintegrants, 5% starch by weight, may be added to the powder blends in the dry state. If more rapid disintegration is desired, this amount may be increased to 10 or 15%. Sodium starch glycolate at 2 to 4% swells 7-fold to 12-fold in less than 30 seconds and Croscarmelose swells 4-fold to 8-fold in less than 10 seconds.

The evolution of carbon dioxide is an effective way to cause fast dissolution of dried forms derived from the solution formulations of bile acid compositions. Dried forms containing a mixture of sodium bicarbonate and an acidulant such as tartaric or citric acid will effervesce when added to water. The amount of sodium bicarbonate may be about ten times the amount of bile acid. The amount of acidulant may be twenty percent more than the amount of sodium bicarbonate. Sufficient acid is added to produce a neutral or slightly acidic reaction when dissolution in water is rapid and complete.

The invention further relates to the preparation of solution formulations derived from bile acid compositions. High throughput sonication with or without heating at about 60° C. may be useful in solubilizing dry or solid preparations of the invention. A high throughput sonication system may be used to drive precipitated compounds back into solution during preparation of solution formulations. The effects of sonication time, power, and amplitude have been optimized in order to drive compounds back into solution. Sonicator that generate sound energy at 20 kHz from 0-1150 watts may be used in forming clear aqueous solutions of the invention.

Dried forms may be prepared from parent solutions by wet granulation, dry granulation and fluid-bed granulation. When ingredients have sufficient inherent binding or cohesive properties, dry granulation method (slugging) may be used to make granules. The general steps of wet and dry granulation are weighing, mixing, granulation (slugging), and screening. Fluid bed granulation may be performed by spraying a granulating solution or solvent into or onto the bed of suspended particles, followed by rapid drying in suspending air. In these systems, suspended particles, which are dried forms derived from parent solutions, may be coated with granulating solution or solvent which contains enteric polymers. The enteric polymers may comprise cellulose acetate phthalate (CAP), which is capable of functioning effectively as an enteric coating at pH greater than 6, polyvinyl acetate phthalate (PVAP), methacrylic acid-methacylic acid ester copolymers, cellulose acetate trimellitate (CAT), carboxymethyl ethylcellulose (CMEC), and hydroxylpropyl methylcellulose acetate succinate (HPMCAS). This granulated form with those enteric polymers remains intact in the stomach but will dissolve and release the active ingredient once it reaches the intestine and colon.

Spheronization, a form of pelletization, refers to the formation of spherical particles (spheres) from wet granulation or fluid bed granulation. Rod shaped cylindrical segments ranging in diameter from 500 microns to 12 millimeters may be prepared through an extruding machine. After extrusion the segments are placed into the Marumerizer where they are shaped into spheres by centrifugal and frictional forces on a rotating plate. The pellets are dried and then coated. In some embodiments of the invention, dried forms of the solution formulations of bile acid compositions may be prepared by spheronization process and then coated with the enteric polymers.

The selected pH range for which a formulation will not precipitate its bile acid, starch conversion product, soluble non-starch polysaccharide or its pharmaceutical compound may be any range of pH levels obtainable with an aqueous system. Preferably this range is between about pH 1 and about pH 14 and more preferably between about pH 1 and about pH 10. Still more preferably the range is any subset of the range of pH levels obtainable in an aqueous system sufficient for the pharmaceutical formulation to remain in solution from preparation, to administration, to absorption in the body, according to the method of administration. In some embodiments of the invention, a bile acid remains dissolved under acidic conditions as a free bile acid in spite of the general insolubility of bile acids under acidic conditions. In some embodiments of the invention, the composition may be used as a pharmaceutical formulation wherein the pharmaceutical compound remains in solution without precipitation at prevailing pH levels in the mouth, stomach or intestines.

The invention contemplates the use of a broad range of pharmaceutical materials. Non-limiting examples include hormones, hormone antagonists, analgesic, antipyretics, antiinflammatory drugs, immunoactive drugs, antineoplastic drugs, antibiotics, anti-inflammatory agents, sympathomimetic drugs, anti-infective drugs, anti-tumor agents, and anesthetics. Further non-limiting examples include drugs that target or effect the gastrointestinal tract, liver, cardiovascular system, and respiratory system. Further non-limiting examples of pharmaceutical compounds include insulin, riluzole, heparin, calcitonin, ampicillin, octreotide, sildenafil citrate, calcitriol, dihydrotachysterol, ampomorphine, yohimbin, trazodone, acyclovir, amantadine.HCl, rimantadine.HCl, cidofovir, delavirdine.mesylate, didanosine, famciclovir, forscamet sodium, fluorouracil, ganciclovir sodium, idoxuridine, interferon-α, interferon-β, interferon-γ, lamivudine, nevirapine, penciclovir, ribavirin, stavudine, trifluridine, valacyclovir.HCl, zalcitabine, zidovudine, indinavir.H₂SO₄, ritonavir, nelfinavir.CH₃SO₃H, saquinavir.CH₃SO₃H, d-penicillamine, chloroquine, hydroxychloroquine, aurothioglucose, gold sodium thiomalate, auranofin levamisole, DTC, isoprinosine, methyl inosine monophosphate, muramyl dipeptide, diazoxide, hydralazine.HCl, minoxidil, dipyridamole, isoxsuprine.HCl, niacin, nylidrin.HCl, phentolamine, doxazosin.CH₃SO₃H, prazosin.HCl, terazocin.HCl, clonidine.HCl, nifedipine, molsidomine, amiodarone, acetylsalicylic acid, verapamil, diltiazem, nisoldipine, isradipine, bepridil, isosorbide.dinitrate, pentaerythrytol.tetranitrate, nitroglycerin, cimetidine, famotidine, nizatidine, ranitidine, lansoprazole, omeprazole, misoprostol, sucralfate, metoclopramide.HCl, erythromycin, bismuth compound, alprostadil, albuterol, pirbuterol, terbutaline.H₂SO₄, salmetrol, aminophylline, dyphylline, ephedrine, ethylnorepinephrine, isoetharine, isoproterenol, metaproterenol, n.docromil, oxy triphylline, theophylline, bitolterol, fenoterol, budesonide, flunisolide, beclomethasone.dipropionate, fluticasone.propionate, codeine, codeine sulfate, codeine phosphate, dextromethorphan.HBr, triamcinolone.acetonide, montelukast sodium, zafirlukast, zileuton, cromolyn sodium, ipratropium bromide, nedocromil sodium benzonate, diphenhydramine.HCl, hydrocodone.bitartarate, methadone.HCl, morphine sulfate, acetylcysteine, guaifenesin, ammonium carbonate, ammonium chloride, antimony potassium tartarate, glycerin, terpin.hydrate, colfosceril palmitate, atorvastatin.calcium, cervastatin.sodium, fluvastatin.sodium, lovastatin, pravastatin.sodium, simvastatin, picrorrhazia kurrva, andrographis paniculata, moringa oleifera, albizzia lebeck, adhata vasica, curcuma longa, momordica charantia, gymnema sylvestre, terminalia arjuna, azadirachta indica, tinosporia cordifolia, metronidazole, amphotericin B, clotrimazole, fluconazole, haloprogin, ketoconazole, griseofulvin, itraconazole, terbinafin.HCl, econazole.HNO₃, miconazole, nystatin, oxiconazole.HNO₃, sulconazole.HNO₃, cetirizine.2HCl, dexamethasone, hydrocortisone, prednisolone, cortisone, catechin and its derivatives, glycyrrhizin, glycyrrhizic acid, betamethasone, ludrocortisone.acetate, flunisolide, fluticasone.propionate, methyl prednisolone, somatostatin, lispro, glucagon, proinsulin, insoluble insulins, acarbose, chlorpropamide, glipizide, glyburide, metformin.HCl, repaglinide, tolbutamide, amino acid, colchicine, sulfinpyrazone, allopurinol, piroxicam, tolmetin sodium, indomethacin, ibuprofen, diflunisal, mefenamic acid, naproxen, and trientine, vitamin E, vitamin C, superoxide dismutase (SOD), N-acetylcysteine, 21-aminosteroid such as lazaroids, U74389F and U74006F, catalase (CAT), putrescine-modified catalase (PUT-CAT), estrogen, alpha-lipoic acid, selegiline, desferrioxanmine, d,1-penicillamine, alpha and beta-carotene, retinol, selenium, gingko biloba, riluzole, flupirtine, pifithrin-alpha, CGP 3466B/TCH346, CPI-1189, CEP-1347, and coenzyme Q10.

Bile acid compositions of the invention may also comprise ginseng. Ginseng contains vitamins A, B-6 and the mineral Zinc, which aids in the production of thymic hormones, necessary for the functioning of the defense system. The main active ingredients of ginseng are the more than 25 saponin triterpenoid glycosides called “ginsenosides.” These steroid-like ingredients provide the adaptogenic properties that enable ginseng to balance and counter the effects of stress. The glycosides appear to act on the adrenal glands, helping to prevent adrenal hypertrophy and excess corticosteroid production in response to physical, chemical or biological stress. Pharmacological effects of ginseng have been demonstrated in the CNS and in cardiovascular, endocrine, and immune systems. In addition, ginseng and its constituents have been ascribed antineoplastic, antistress, and antioxidant activity. It is an herb with many active components, and there is evidence from numerous studies that ginseng does have beneficial effects. Ginseng has demonstrated the combined effects with various oriental medicines to increase intensity of response or efficacy, to decrease individual toxicity, to antagonize untoward actions and to alter absorption for long periods.

Pharmacological effects of ginseng have been demonstrated in the CNS and in cardiovascular, endocrine, and immune systems. In addition, ginseng and its aqueous soluble constituents have been ascribed antineoplastic, anti-stress, and antioxidant activity. In some embodiments of the invention, solution formulations may comprise aqueous soluble ginseng (white and red) extract.

Thus, the invention contemplates the use of a broad range of pharmaceutical materials. Any pharmaceutical material that becomes and/or remains soluble in formulations of the invention may be used. With an additional pharmaceutical compound in the formulation, a bile acid in solution may act as an adjuvant, carrier, or enhancer for the solubility of certain therapeutically active agents, including, but not limited to, insulin (pH 7.4-7.8), heparin (pH 5-7.5), calcitonin, ampicillin, amantadine, rimantadine, sildenafil, neomycin sulfate (pH 5-7.5), apomorphine, yohimbin, trazodone, ribavirin, paclitaxel and its derivatives, retinol, and tretinoin, which are soluble and stable in acid and/or alkali and can be added as needed into these aqueous solution formulations of certain concentrations of bile acids in this invention. Certain therapeutically active agents, including, but not limited to, metformin HCl (pH 5-7), ranitidine HCl, cimetidine, lamivudine, cetrizine 2HCl (pH 4-5), amantadine, rimantadine, sildenafil, apomorphine, yohimbine, trazodone, ribavirin and dexamethasone, hydrocortisone, prednisolone, triamcinolone, cortisone, niacin, taurine, vitamins, naturally occurring amino acids, catechin and its derivatives, glycyrrhizal extract and its main constituents such as glycyrrhizin and glycyrrhizic acid, water soluble bismuth compounds (e.g., bismuth sodium tartrate), and which are soluble and stable in acid and/or alkali can be added as needed into these aqueous solution dosage formulations containing ursodeoxycholic acid in this invention.

According to the invention bismuth compounds comprise an aqueous soluble reaction product between a bismuth ion and a chelator. Non-limiting examples of such chelators include citric acid, tartaric acid, malic acid, lactic acid and eidetic acid. Non-limiting examples include of bismuth citrate, bismuth sulfate, bismuth subnitrate, bismuth subcarbonate, bismuth subsalicylate, and bismuth gallate.

The invention contemplates the use of pH adjustable agents. Non-limiting examples include HCl, H₂SO₄, HNO₀₃, CH₃COOH, citric acid, malic acid, tartaric acid, lactic acid, phosphate, eidetic acid and alkalies.

In some embodiments of the invention, the formulations may be used to treat human and mammalian diseases. The invention contemplates treating gastrointestinal disorders, liver diseases, gall stones, and hyperlipidemia. Non-limiting examples of liver diseases include alcohol-induced liver diseases and non-alcohol-induced liver diseases. Non-limiting examples of gastrointestinal disorders include chronic gastritis, reflux gastritis, and peptic ulcer disease. Non-limiting examples of non-alcohol-induced liver diseases include primary biliary cirrhosis, acute and chronic hepatitis, primary sclerosing cholangitis, chronic active hepatitis, and excess accumulation of fat in the liver. The invention further contemplates treating viral, bacterial and fungal diseases. In some embodiments of the invention, a formulation is administered to treat and/or eradicate Helicobacter pylori infection. In some embodiments of the invention, a formulation is administered to treat and/or eradicate hepatitis C virus infection, influenza A, Influenza C, parainfluenza 1, sendai, rubella, and pseudorabies virus. In some embodiments of the invention, a formulation is administered to treat acute or chronic inflammatory diseases. Non-limiting examples of inflammatory diseases include bronchitis, chronic pharyngitis, and chronic tonsillitis. In some embodiments of the invention, a formulation is administered to treat hypercholersterolemia.

In some embodiments of the invention, the formulation is modified such that it may be administered as a liquid, solid, powder or tablet. In some embodiments of the invention, the formulation is comprised in a syrup, thick syrup or paste. A non-limiting example of a syrup is a solution of maltodextrin wherein the concentration of maltodextrin is less than 1.0 kg/L. A non-limiting example of thick syrup is a solution of maltodextrin wherein the concentration of maltodextrin is between 1.0 kg/L and 1.2 kg/L inclusive. A non-limiting example of a paste is a solution of maltodextrin wherein the concentration of maltodextrin is greater than 1.2 kg/L.

The aqueous solutions of the invention may be dried. For the purpose of this disclosure, a “primary” aqueous solution bile acid dosage formulation according to the invention is produced by the original combination of a bile acid or its salts and a carbohydrate with water. It may be prepared by a simultaneous or stepwise combination of ingredients. A “secondary” aqueous solution bile acid dosage formulation, by contrast, is a solution prepared from a powder or solid comprising previously co-dissolved bile acid and carbohydrate. Thus, a secondary aqueous solution bile acid dosage formulation differs at least in that water has been added, removed, and added again.

In some embodiments of the invention, a primary aqueous solution bile acid dosage formulation is dried by spray-drying. Spray-drying consists of bringing together a highly dispersed liquid and a sufficient volume of hot air to produce evaporation and drying of the liquid droplets. The feed liquid may be a solution, slurry, syrup or paste provided it is pumpable and capable of being atomized. The liquid feed is sprayed into a current of warm filtered air. The air supplies the heat for evaporation and conveys the dried product to the collector; the air is then exhausted with the moisture. The spray-dried powder particles are homogeneous, approximately spherical in shape, nearly uniform in size, and frequently hollow. The latter characteristic results in low bulk density with a rapid rate of solution. This process is useful in coating one material on another to protect the interior substance or to control the rate of its release. For example, dried form of an aqueous solution bile acid dosage formulation can be coated with enteric polymers for the colonic delivery of solubilized UDCA. Dehydration may also be accomplished with lyophilization, evaporation or any other dehydration technique known in the art.

The resulting dried form, e.g. powder or solid, may be administered directly or recombined with water to produce a secondary clear aqueous solution bile acid dosage formulation. Secondary aqueous solution bile acid dosage formulations, i.e. those produced from dried forms, have substantially the same properties as primary formulations.

The invention contemplates the addition of additives such as pharmaceuticals to primary and secondary aqueous bile acid solutions as well as to dried forms. If administered in dried form, the dried material may be combined with one or more diluents, lubricants, binders, fillers, drugs, disintegrants or other additives. Thus, the dried form may be comprised in a powder, granule, a pill, tablet or capsule.

The stability of dosage formulations of the invention were evaluated by measuring the concentration of the relevant bile acid over time in preparations comprising soluble bile acid, a high molecular weight aqueous soluble starch conversion product, and water at various pH and temperature levels. The stability tests were performed with HPLC and microscope light at various pH conditions under the normal and accelerated conditions. Solution stability tests included concentration analyses for each bile acid, performed by HPLC as follows: the elution solvent was 0.02 M KH₂PO₄:acetonitrile in a ratio of 55:45, with a pH of 3.01; the flow rate was 0.8 milliliters/minute; the injection volume was 20 μL, and the detection wave length was 195 nm. The retention time of each bile acid may be adjusted as needed to permit individual analysis of each bile acid present in a sample having a plurality of bile acids. In the tables, the concentration of the indicated bile acid salt for each of the three numbered trials and the average thereof is reported on each line. The percentage indicates the relative concentration of the bile acid salt after incubation for a certain amount of time in comparison with the initial concentration.

Accelerated conditions for testing pharmaceutical compositions have been described (Remington, The Science and Practice of Pharmacy, 19th ed., p. 640). All of these stability test results were satisfactory in that the concentration of bile acid as measured by HPLC did not change appreciably over time at various pH levels. Thus, the formulations of the examples are suitable for preparing a commercial liquid dosage form. Particularly, all solution formulations which contained bile acid showed excellent results in the stability tests with no precipitation and no physical appearance changes over the test period. Some formulations remain stable for over 2 years.

Stability tests were also conducted on the aqueous solution formulations comprising a mixture of aqueous soluble UDCA, branched chained amino acid (leucine, isoleucine, valine) and maltodextrin according to example IV. This formulation may be typical of solution formulations in which bile acid functions as a therapeutically active agent, as an adjuvant or carrier, pharmaceutically active agent, and a solubility enhancer. According to the test results, there were no clarity changes, no discoloration, and no precipitation. Furthermore, there are no detectable impurities from the deterioration of UDCA or branched chained amino acids when examined by HPLC at various pH conditions such as pH 1, 3, 5, 7, 9, and 10 under the accelerated conditions (e.g. incubation at 50° C.).

The aqueous solution formulations according to this invention did not change either physically or chemically at various pH conditions under the accelerated conditions despite the addition of therapeutically and chemically active agents that are stable and soluble in hydrochloric acid solution. Therefore, these aqueous solution systems may be extremely valuable pharmaceutical dosage forms for delivery of therapeutically active bile acids, and/or drugs. Without being limited to any particular mode of action, in drug (pharmaceutical compound) delivery preparations, bile acids may function as an adjuvant, a carrier, or a solubility enhancer (e.g. by micelle formation).

### EXAMPLES

Stability tests were conducted on three different aqueous solution systems. First, a bile acid and a high molecular weight aqueous soluble starch conversion product were combined in aqueous solution according to Example I and tested. Results are shown in Tables 1A and 1B. Second, mixed bile acids and high molecular weight aqueous soluble starch conversion products were combined in aqueous solution according to Example II and tested. Results are shown in Table 2. Third, bile acids, high molecular weight aqueous soluble starch conversion products and branched chained amino acids (e.g. leucine, isoleucine, valine, or other amino acid with a branched side chain) were combined in aqueous solution according to Example IV and tested. Results are shown in Tables 3A through 3F.

### Example I

A first series of solution formulations that were prepared with soluble bile acids (as free acids) and high molecular weight aqueous soluble starch conversion products according to the following guidelines did not show any precipitation at any pH tested.

| | Starch Conversion | |
|---|---|---|
| Soluble Bile Acid | Product (Minimum) | |
| if 200 mg CDCA | about 30 | g |
| if 200 mg UDCA | about 5 | g |
| if 200 mg KLCA | about 12 | g |
| if 200 mg cholic acid | about 10 | g |
| if 200 mg deoxycholic acid | about 50 | g |
| if 200 mg hyodeoxycholic acid | about 3.5 | g |
| Purified water to make | 100 | mL |

Aqueous solutions (100 mL) in which one of the above soluble bile acids is dissolved were prepared. Maltodextrin, as one high molecular weight aqueous soluble starch conversion product, was dissolved with agitation about 60-80° C. to make a clear solution. The pH of this resulting clear solution was adjusted by acid to prepare oral dosage forms, topical preparations, and solutions. Purified water was added to make the total volume be 100 mL. According to the instant invention and all examples, purified water is deionized, distilled deionized-distilled water, or any grade commonly used for pharmaceutical preparations.

Based on these formulas, aqueous solution formulations with various concentrations of certain bile acids (or salts) and their corresponding minimal quantities of high molecular weight aqueous soluble starch conversion products that have a DE of 15-25 (e.g. Maltrin®M150 (DE=15), Maltrin®M180 (DE=18), Maltrin®M200 (DE=20), Maltrin®M250 (corn syrup solid; DE=25), liquid glucose) or soluble non-starch polysaccharides (e.g. guar gum, pectin, gum arabic) were prepared.

### Example II

A second series of solution formulations that were prepared with soluble bile acids (as free acids) and high molecular weight aqueous soluble starch conversion products according to the following guidelines did not show any precipitation at any pH tested.

| | Starch Conversion | |
|---|---|---|
| Soluble Bile Acid | Product (Minimum) | |
| if 200 mg CDCA | about 18 | g |
| if 200 mg UDCA | about 3 | g |
| if 200 mg KLCA | about 7.2 | g |
| if 200 mg cholic acid | about 6 | g |
| if 200 mg deoxycholic acid | about 30 | g |
| if 200 mg hyodeoxycholic acid | about 2.1 | g |
| Purified water to make | 100 | mL |

Aqueous solutions (100 mL) in which one of the above soluble bile acids is dissolved were prepared. Maltodextrin, as one high molecular weight aqueous soluble starch conversion product, was added to the resulting solution and dissolved with agitation at room temperature to make a clear solution. The pH of this resulting clear solution was adjusted by acid with high throughput sonication to prepare oral dosage forms, topical preparations, and solutions. Purified water was added to make the total volume be 100 mL.

Based on these formulas, aqueous solution formulations with various concentrations of certain bile acids (or salts) and their corresponding minimal quantities of high molecular weight aqueous soluble starch conversion products that have a DE of 5-10 (e.g. Maltrin®M040, Maltrin®M 100) or soluble non-starch polysaccharides (e.g. guar gum, pectin, gum arabic) were prepared.

### Example III

A third series of solution formulations that were prepared with soluble bile acids (as free acids) and high molecular weight aqueous soluble starch conversion products according to the following guidelines did not show any precipitation at pH 6.5-8.

| | Starch Conversion | |
|---|---|---|
| Soluble Bile Acid | Product (Minimum) | |
| if 200 mg CDCA | about 15 | g |
| if 200 mg UDCA | about 1.5 | g |
| if 200 mg KLCA | about 3.6 | g |
| if 200 mg cholic acid | about 3 | g |
| if 200 mg deoxycholic acid | about 15 | g |
| if 200 mg hyodeoxycholic acid | about 3.5 | g |
| Purified water to make | 100 | mL |

Aqueous solutions (100 mL) in which one of the above soluble bile acids is dissolved were prepared. Maltodextrin, as one high molecular weight aqueous soluble starch conversion product, was added to the resulting solution and dissolved with agitation at room temperature to make a clear solution. The pH of this resulting clear solution was adjusted by acid with high throughput sonication to prepare injectable, colon-specific, topical, and eye drops dosage forms. Purified water or water for injection was added to make the total volume be 100 mL.

Based on these formulas, aqueous solution formulations with various concentrations of certain bile acids (or salts) and their corresponding minimal quantities of high molecular weight aqueous soluble starch conversion products that have a DE of 5-10 (e.g. Maltrin®M040, Maltrin®M100) or soluble non-starch polysaccharides (e.g. guar gum, pectin, gum arabic) were prepared.

### Example IV

A fourth series of solution formulations that were prepared with soluble bile acids (as free acids), high molecular weight aqueous soluble starch conversion products, and non-starch polysaccharides according to the following guidelines did not show any precipitation at any pH tested.

| | |
|---|---|
| Soluble Bile Acid | 200 mg KLCA (10 mg to 3 g) |
| Starch Conversion Product (Minimum) | about 24 g (0.6 g to 75 g) |
| Soluble Fiber | 20 g (5 g to 30 g) |
| Purified water to make | 100 mL |

Aqueous solutions (60 mL) in which soluble KLCA is dissolved were prepared. Maltodextrin, as one high molecular weight aqueous soluble starch conversion product, was added to the resulting solution and dissolved with agitation at room temperature to make a clear solution. The pH of this resulting clear solution was adjusted by acid with high throughput sonication to prepare injectable and topical dosage forms. Next, soluble non-starch polysaccharide (e.g. guar gum, pectin, gum arabic) and soluble resistant maltodextrin was added. Purified water or water for injection was added to make the total volume be 100 mL.

Table IA shows the results of a test of stability over time at pH 7 and 50° C. of formulations of CA, 7-ketolithocholic acid, CDCA and DCA in solution with maltodextrin prepared according to Example I. The concentrations of the bile acids were measured by HPLC and the concentration of the bile acid as a percentage of its concentration on day 0 is reported in the column labeled percentage.

Table 1B shows the results of the test of stability over time at pH 10 and 50° C. of formulations of CA, 7-ketolithocholic acid, CDCA and DCA in solution with maltodextrin prepared according to Example I.

Table 2 shows results of the test of stability over time at pH 1 and 50° C. of formulations of CA, 7-ketolithocholic acid, CDCA and DCA in solution with maltodextrin at pH 1 and 50° C. prepared according to Example II.

### Example V

A fifth series of solution formulations that were prepared with soluble bile acids (as free base), high molecular weight aqueous soluble starch conversion products, and non-starch polysaccharides according to the following guidelines did not show any precipitation at

| | |
|---|---|
| Soluble Bile Acid | 200 mg UDCA (10 mg to 3 g) |
| Starch Conversion Product (Minimum) | about 5 g (0.25 g to 75 g) |
| Resistant maltodextrin | 15 g (5 g to 30 g) |
| Purified water to make | 100 mL |

Aqueous solutions (100 mL) in which soluble UDCA is dissolved were prepared. Maltodextrin, as one high molecular weight aqueous soluble starch conversion product, was added to the resulting clear solution and dissolved with agitation at room temperature to make a clear solution. The pH of this resulting clear solution was adjusted by acid with high throughput sonication to prepare oral and topical dosage forms. Next, soluble resistant maltodextrin was added. Purified water or water for injection was added to make the total volume be 100 mL.

Based on these formulas, aqueous solution formulations with various concentrations of UDCA (or its salts) and their corresponding minimal quantities of high molecular weight aqueous soluble starch conversion products that have a DE of 5-40 (e.g. Maltrin®M150, Maltrin®M180, Maltrin®M200, Maltrin®M250, Maltrin®M040, Maltrin®M100) and soluble resistant maltodextrin were prepared.

### Example VI

A sixth series of solution formulations that were prepared with soluble bile acids (as free base), high molecular weight aqueous soluble starch conversion products, and ginseng extract according to the following guidelines did not show any precipitation at any pH tested.

| | |
|---|---|
| Soluble Bile Acid | 200 mg UDCA (10 mg to 3 g) |
| Starch Conversion Product (Minimum) | about 5 g (0.25 g to 75 g) |
| Aqueous Soluble Ginseng Extract | 200 mg (50 mg to 3 g) |
| Purified water to make | 100 mL |

Aqueous solutions (80 mL) in which soluble UDCA is dissolved were prepared. Maltodextrin, as one high molecular weight aqueous soluble starch conversion product, was added to the resulting clear solution and dissolved with agitation at room temperature to make a clear solution. The pH of this resulting clear solution was adjusted by acid with high throughput sonication to prepare oral and topical dosage forms. Next, soluble ginseng extract was added. Purified water or water for injection was added to make the total volume be 100 mL.

Based on these formulas, aqueous solution formulations with various concentrations of UDCA (or its salts) and its corresponding minimal quantities of high molecular weight aqueous soluble starch conversion products that have a DE of 5-40 were prepared. Aqueous soluble ginseng extract comprises extract from red ginseng and white ginseng.

### Example VII

A seventh series of solution formulations that were prepared with soluble bile acids (as free base), high molecular weight aqueous soluble starch conversion products, and ginseng extract according to the following guidelines did not show any precipitation at any pH tested.

| | |
|---|---|
| Soluble Bile Acid | 200 mg UDCA (10 mg to 3 g) |
| Starch Conversion Product (Minimum) | about 5 g (0.25 g to 75 g) |
| Aqueous Soluble Ginseng Extract | 200 mg (50 mg to 5 g) |
| Soluble Non-Starch Polysaccharide | 5-20 g |
| Purified water to make | 100 mL |

Aqueous solutions (80 mL) in which soluble UDCA is dissolved were prepared. Maltodextrin, as one high molecular weight aqueous soluble starch conversion product, was added to the resulting clear solution and dissolved with agitation at room temperature to make a clear solution. The pH of this resulting clear solution was adjusted by acid with high throughput sonication to prepare oral and topical dosage forms. Then, aqueous soluble ginseng extract and soluble non-starch polysaccharide (e.g. guar gum, pectin, gum arabic) or soluble resistant maltodextrin were added. Purified water or water for injection was added to make the total volume be 100 mL.

Based on these formulas, aqueous solution formulations with various concentrations of UDCA (or its salts) and its corresponding minimal quantities of high molecular weight aqueous soluble starch conversion products that have a DE of 5-40 were prepared. Aqueous soluble ginseng extract comprises extract from red ginseng and white ginseng. Soluble fiber is soluble non-starch polysaccharide (e.g. guar gum, pectin, gum arabic) or soluble resistant maltodextrin.

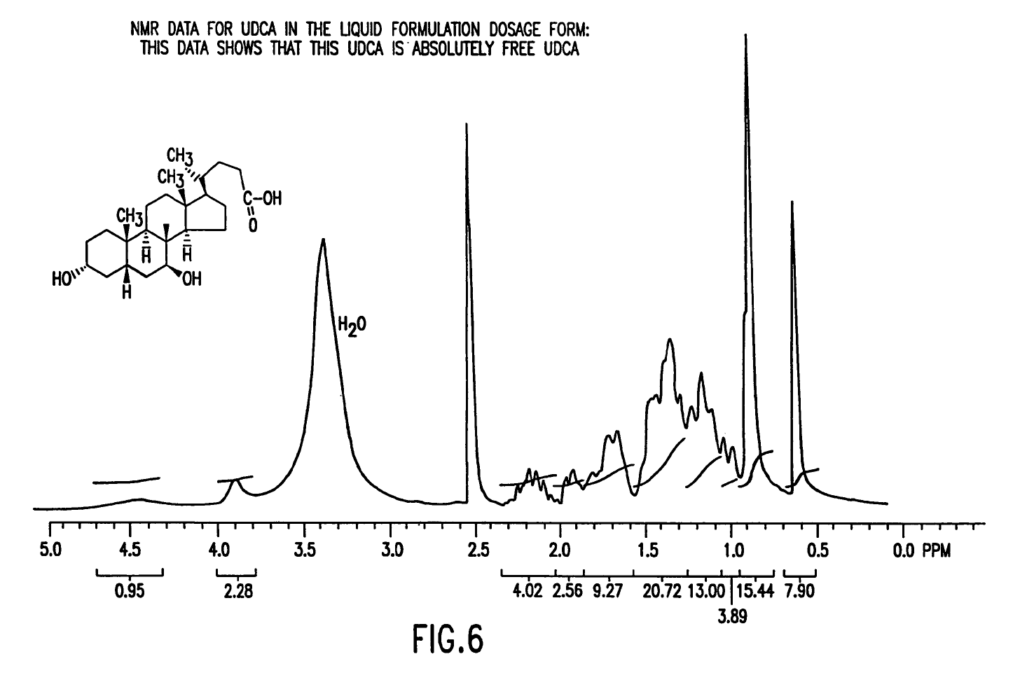

FIG. 6 is an NMR spectrum of UDCA illustrating that UDCA, when in a composition prepared according to Example III, is absolutely free UDCA. That is, the carboxylic acid of UDCA at C-24 is the free form (R—COOH) and two hydroxy group at C-3 and C-7 are in the free form (R—OH).

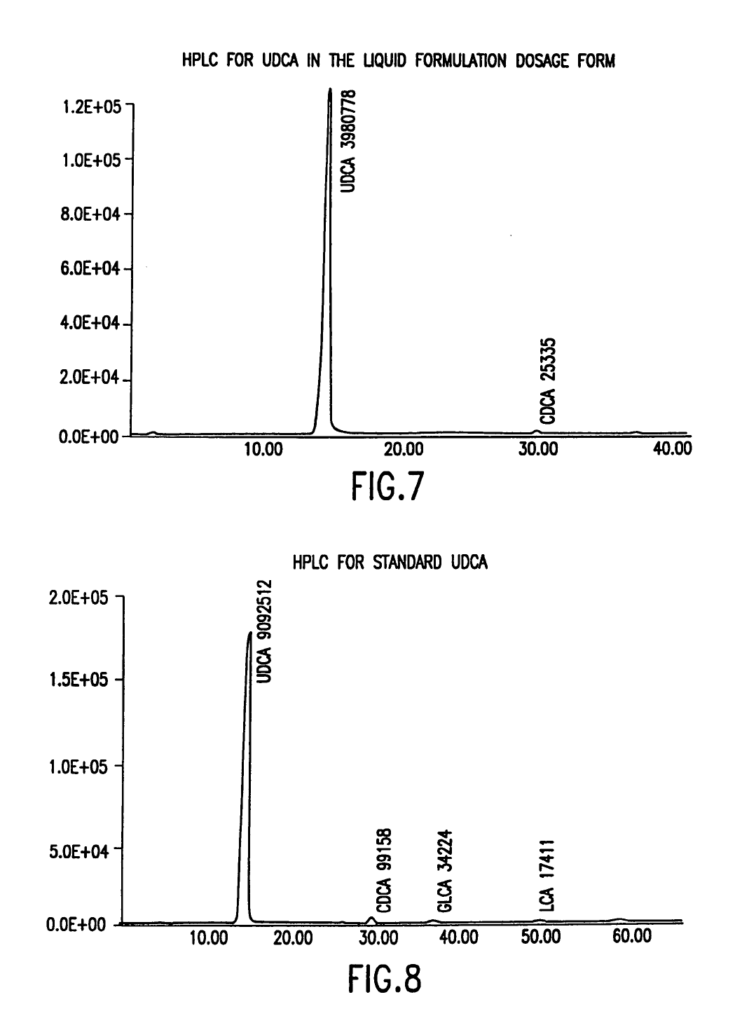

In addition, the HPLC profile of UDCA in a composition prepared according to Example III (FIG. 7) is similar to the profile of UDCA dissolved in methanol (FIG. 8). This data shows that there is no UDCA-complex compound. There is only free UDCA. A non-aqueous UDCA standard solution was prepared by dissolving 100 mg UDCA in 100 mL of methanol. A mixture of acetonitrile (51), water (49), and acetic acid (1) was used as the mobile phase.

### Example VIII

An eighth series of solution formulations that were prepared with soluble bile acids (as free acid), high molecular weight aqueous soluble starch conversion products, and branched chain amino acids (e.g. leucine, isoleucine, valine) according to the following guidelines did not show any precipitation at any pH tested.

| | |
|---|---|
| Soluble Bile Acid | 200 mg UDCA (10 mg to 3 g) |
| Starch Conversion Product (Minimum) | about 5 g (0.25 g to 75 g) |
| Branched Chained Amino Acid | 15 g (1 g to 35 g) |
| Purified water to make | 100 mL |

Aqueous solutions (85 mL) in which soluble UDCA is dissolved were prepared. Maltodextrin, as one high molecular weight aqueous soluble starch conversion product, was added to the resulting clear solution and dissolved with agitation at room temperature to make a clear solution. The pH of this resulting clear solution was adjusted (to from pH 4 to pH 7) by acid with high throughput sonication to prepare oral and topical dosage forms. Then, branched chain amino acids were added. Purified water or water for injection was added to make the total volume be 100 mL.

Based on these formulas, aqueous solution formulations with various concentrations of UDCA (or its salts) and its corresponding minimal quantities of high molecular weight aqueous soluble starch conversion products that have a DE of 5-40 were prepared with various quantities of branched amino acid (total amount of leucine, isoleucine and valine).

Tables 3A to 3F show stability test results over time of formulation prepared with amino acids according to Example IV. All stability tests were conducted at 50 C. Stability test results at pH 1 (Table 3A), pH 3 (Table 3B), pH 5 (Table 3C), pH 7 (Table 3D), pH 9 (Table 3E), and pH 10 (Table 3F) are shown.

TABLE 3A
Stability of UDCA solution according to Example IV at pH 1, 50 C.

| | Day | #1 | #2 | #3 | Average | Percentage |
|---|---|---|---|---|---|---|
| Ile | 0 | 0.261 | 0.236 | 0.249 | 0.248 | 100.0 |
| | 1 | 0.256 | 0.275 | 0.251 | 0.261 | 105.0 |
| | 2 | 0.268 | 0.263 | 0.251 | 0.260 | 104.9 |
| | 6 | 0.295 | 0.268 | 0.291 | 0.285 | 114.6 |
| | 7 | 0.249 | 0.254 | 0.267 | 0.257 | 103.4 |
| | 8 | 0.253 | 0.243 | 0.240 | 0.245 | 98.8 |
| | 9 | 0.263 | 0.268 | 0.263 | 0.265 | 106.6 |
| Leu | 0 | 0.485 | 0.428 | 0.470 | 0.461 | 100.0 |
| | 1 | 0.470 | 0.477 | 0.456 | 0.468 | 101.5 |
| | 2 | 0.485 | 0.481 | 0.460 | 0.475 | 103.1 |
| | 6 | 0.553 | 0.510 | 0.529 | 0.531 | 115.1 |
| | 7 | 0.478 | 0.473 | 0.513 | 0.488 | 105.8 |
| | 8 | 0.474 | 0.454 | 0.511 | 0.480 | 104.0 |
| | 9 | 0.483 | 0.485 | 0.476 | 0.481 | 104.4 |
| Val | 0 | 0.506 | 0.448 | 0.460 | 0.471 | 100.0 |
| | 1 | 0.438 | 0.458 | 0.471 | 0.456 | 96.7 |
| | 2 | 0.479 | 0.485 | 0.513 | 0.492 | 104.5 |
| | 6 | 0.505 | 0.536 | 0.549 | 0.530 | 112.4 |
| | 7 | 0.494 | 0.465 | 0.496 | 0.485 | 102.9 |
| | 8 | 0.488 | 0.491 | 0.459 | 0.479 | 101.7 |
| | 9 | 0.479 | 0.496 | 0.490 | 0.488 | 103.6 |
| Sol | 0 | 0.319 | 0.315 | 0.322 | 0.319 | 100.0 |
| | 1 | 0.332 | 0.344 | 0.351 | 0.342 | 107.4 |
| | 2 | 0.371 | 0.339 | 0.403 | 0.371 | 116.4 |
| | 6 | 0.396 | 0.409 | 0.411 | 0.405 | 127.2 |
| | 7 | 0.365 | 0.351 | 0.381 | 0.366 | 114.7 |
| | 8 | 0.409 | 0.365 | 0.331 | 0.368 | 115.6 |
| | 9 | 0.338 | 0.391 | 0.374 | 0.368 | 115.4 |
| UDCA | 0 | 0.388 | 0.387 | 0.389 | 0.388 | 100.0 |
| | 1 | 0.367 | 0.370 | 0.366 | 0.368 | 94.8 |
| | 2 | 0.374 | 0.388 | 0.388 | 0.383 | 98.9 |
| | 6 | 0.371 | 0.380 | 0.382 | 0.377 | 97.3 |
| | 7 | 0.378 | 0.376 | 0.379 | 0.378 | 97.4 |
| | 8 | 0.374 | 0.382 | 0.384 | 0.380 | 97.9 |
| | 9 | 0.370 | 0.367 | 0.370 | 0.369 | 95.1 |

TABLE 3B
Stability of UDCA solution according to Example IV at pH 3, 50 C.

| | Day | #1 | #2 | #3 | Average | Percentage |
|---|---|---|---|---|---|---|
| Ile | 0 | 0.261 | 0.254 | 0.253 | 0.256 | 100.0 |
| | 1 | 0.266 | 0.268 | 0.261 | 0.265 | 103.3 |
| | 2 | 0.273 | 0.243 | 0.247 | 0.254 | 99.3 |
| | 6 | 0.296 | 0.306 | 0.300 | 0.301 | 117.4 |
| | 7 | 0.247 | 0.265 | 0.257 | 0.256 | 100.0 |
| | 8 | 0.250 | 0.247 | 0.247 | 0.248 | 96.7 |
| | 13 | 0.285 | 0.240 | 0.250 | 0.258 | 100.9 |
| Leu | 0 | 0.495 | 0.465 | 0.452 | 0.471 | 100.0 |
| | 1 | 0.489 | 0.480 | 0.470 | 0.480 | 101.9 |
| | 2 | 0.495 | 0.472 | 0.481 | 0.483 | 102.6 |
| | 6 | 0.522 | 0.532 | 0.556 | 0.537 | 114.0 |
| | 7 | 0.492 | 0.482 | 0.491 | 0.488 | 103.7 |
| | 8 | 0.543 | 0.515 | 0.495 | 0.517 | 109.9 |
| | 13 | 0.512 | 0.496 | 0.543 | 0.517 | 109.8 |
| Val | 0 | 0.485 | 0.491 | 0.498 | 0.491 | 100.0 |
| | 1 | 0.467 | 0.481 | 0.446 | 0.465 | 94.6 |
| | 2 | 0.510 | 0.493 | 0.527 | 0.510 | 103.8 |
| | 6 | 0.527 | 0.491 | 0.553 | 0.524 | 106.6 |
| | 7 | 0.485 | 0.481 | 0.468 | 0.478 | 97.3 |
| | 8 | 0.490 | 0.491 | 0.544 | 0.508 | 103.5 |
| | 13 | 0.519 | 0.498 | 0.517 | 0.511 | 104.1 |
| Sol | 0 | 0.343 | 0.355 | 0.370 | 0.356 | 100.0 |
| | 1 | 0.340 | 0.350 | 0.316 | 0.335 | 94.2 |
| | 2 | 0.383 | 0.371 | 0.400 | 0.385 | 108.0 |
| | 6 | 0.378 | 0.341 | 0.416 | 0.378 | 106.3 |
| | 7 | 0.355 | 0.381 | 0.315 | 0.350 | 98.4 |
| | 8 | 0.343 | 0.350 | 0.395 | 0.363 | 101.9 |
| | 13 | 0.377 | 0.382 | 0.423 | 0.394 | 110.7 |
| UDCA | 0 | 0.395 | 0.396 | 0.393 | 0.395 | 100.0 |
| | 1 | 0.396 | 0.401 | 0.392 | 0.396 | 100.4 |
| | 2 | 0.427 | 0.421 | 0.416 | 0.421 | 106.8 |
| | 6 | 0.407 | 0.408 | 0.402 | 0.405 | 102.7 |
| | 7 | 0.412 | 0.409 | 0.411 | 0.411 | 104.1 |
| | 8 | 0.415 | 0.418 | 0.408 | 0.414 | 104.9 |
| | 13 | 0.415 | 0.412 | 0.416 | 0.414 | 105.0 |

TABLE 3C
Stability of UDCA solution according to Example IV at pH 5, 50 C.

| | Day | #1 | #2 | #3 | Average | Percentage |
|---|---|---|---|---|---|---|
| Ile | 0 | 0.285 | 0.258 | 0.295 | 0.279 | 100.0 |
| | 3 | 0.280 | 0.275 | 0.275 | 0.277 | 99.0 |
| | 6 | 0.285 | 0.273 | 0.270 | 0.276 | 98.7 |
| | 10 | 0.274 | 0.276 | 0.276 | 0.275 | 98.4 |
| | 13 | 0.273 | 0.287 | 0.278 | 0.279 | 100.0 |
| | 17 | 0.278 | 0.276 | 0.270 | 0.275 | 98.3 |
| | 20 | 0.261 | 0.275 | 0.261 | 0.266 | 95.0 |
| | 24 | 0.267 | 0.274 | 0.292 | 0.277 | 99.3 |
| Leu | 0 | 0.495 | 0.467 | 0.535 | 0.499 | 100.0 |
| | 3 | 0.510 | 0.495 | 0.494 | 0.500 | 100.1 |
| | 6 | 0.489 | 0.479 | 0.484 | 0.484 | 97.0 |
| | 10 | 0.486 | 0.490 | 0.499 | 0.492 | 98.5 |
| | 13 | 0.492 | 0.509 | 0.508 | 0.503 | 100.8 |
| | 17 | 0.514 | 0.508 | 0.504 | 0.509 | 100.9 |
| | 20 | 0.499 | 0.500 | 0.499 | 0.499 | 101.1 |
| | 24 | 0.488 | 0.509 | 0.528 | 0.508 | 101.9 |
| Val | 0 | 0.483 | 0.498 | 0.481 | 0.487 | 100.0 |
| | 3 | 0.492 | 0.494 | 0.526 | 0.504 | 103.4 |
| | 6 | 0.459 | 0.475 | 0.481 | 0.472 | 96.8 |
| | 10 | 0.500 | 0.436 | 0.480 | 0.472 | 96.9 |
| | 13 | 0.464 | 0.451 | 0.474 | 0.463 | 95.0 |
| | 17 | 0.407 | 0.491 | 0.462 | 0.453 | 93.0 |
| | 20 | 0.471 | 0.512 | 0.477 | 0.487 | 99.9 |
| | 24 | 0.471 | 0.476 | 0.458 | 0.468 | 96.1 |
| Sol | 0 | 0.341 | 0.351 | 0.360 | 0.351 | 100.0 |
| | 3 | 0.342 | 0.386 | 0.371 | 0.366 | 104.5 |
| | 6 | 0.316 | 0.321 | 0.342 | 0.326 | 93.1 |
| | 10 | 0.341 | 0.299 | 0.335 | 0.325 | 92.7 |
| | 13 | 0.355 | 0.326 | 0.350 | 0.344 | 98.0 |
| | 17 | 0.334 | 0.376 | 0.353 | 0.354 | 101.0 |
| | 20 | 0.347 | 0.398 | 0.394 | 0.380 | 108.3 |
| | 24 | 0.416 | 0.353 | 0.378 | 0.382 | 109.0 |
| UDCA | 0 | 0.407 | 0.404 | 0.404 | 0.405 | 100.0 |
| | 3 | 0.409 | 0.402 | 0.403 | 0.405 | 99.9 |
| | 6 | 0.410 | 0.403 | 0.409 | 0.407 | 100.6 |
| | 10 | 0.404 | 0.405 | 0.407 | 0.405 | 100.1 |
| | 13 | 0.408 | 0.403 | 0.395 | 0.402 | 99.3 |
| | 17 | 0.411 | 0.402 | 0.404 | 0.406 | 100.2 |
| | 20 | 0.405 | 0.394 | 0.396 | 0.398 | 98.4 |
| | 24 | 0.399 | 0.408 | 0.406 | 0.404 | 99.9 |

TABLE 3D
Stability of UDCA solution according to Example IV at pH 7, 50 C.

| | | | | | |
|---|---|---|---|---|---|
| Day | #1 | #2 | #3 | Average | Percentage |

| | | | | | | |
|---|---|---|---|---|---|---|
| Ile | 0 | 0.296 | 0.289 | 0.281 | 0.289 | 100.0 |
| | 5 | 0.300 | 0.282 | 0.281 | 0.288 | 99.7 |
| | 8 | 0.277 | 0.282 | 0.268 | 0.276 | 95.5 |
| | 12 | 0.273 | 0.278 | 0.278 | 0.277 | 95.8 |
| | 15 | 0.271 | 0.273 | 0.266 | 0.270 | 93.5 |
| | 19 | 0.294 | 0.285 | 0.281 | 0.287 | 99.3 |
| Leu | 0 | 0.519 | 0.513 | 0.495 | 0.509 | 100.0 |
| | 5 | 0.499 | 0.499 | 0.498 | 0.498 | 97.9 |
| | 8 | 0.498 | 0.513 | 0.480 | 0.497 | 97.7 |
| | 12 | 0.508 | 0.516 | 0.515 | 0.513 | 100.9 |
| | 15 | 0.503 | 0.505 | 0.499 | 0.502 | 98.7 |
| | 19 | 0.521 | 0.509 | 0.516 | 0.515 | 101.3 |
| Val | 0 | 0.483 | 0.530 | 0.525 | 0.513 | 100.0 |
| | 5 | 0.502 | 0.447 | 0.499 | 0.483 | 94.1 |
| | 8 | 0.488 | 0.498 | 0.493 | 0.493 | 96.2 |
| | 12 | 0.490 | 0.469 | 0.443 | 0.467 | 91.2 |
| | 15 | 0.492 | 0.541 | 0.442 | 0.492 | 95.9 |
| | 19 | 0.458 | 0.500 | 0.482 | 0.480 | 93.6 |
| Sol | 0 | 0.333 | 0.352 | 0.363 | 0.349 | 100.0 |
| | 5 | 0.344 | 0.309 | 0.349 | 0.334 | 95.6 |
| | 8 | 0.334 | 0.379 | 0.377 | 0.363 | 104.0 |
| | 12 | 0.345 | 0.344 | 0.317 | 0.335 | 96.0 |
| | 15 | 0.286 | 0.406 | 0.321 | 0.338 | 96.7 |
| | 19 | 0.338 | 0.416 | 0.351 | 0.368 | 105.4 |
| UDCA | 0 | 0.427 | 0.416 | 0.428 | 0.424 | 100.0 |
| | 5 | 0.406 | 0.427 | 0.432 | 0.422 | 99.4 |
| | 8 | 0.419 | 0.408 | 0.417 | 0.414 | 97.7 |
| | 12 | 0.414 | 0.418 | 0.419 | 0.417 | 98.4 |
| | 15 | 0.413 | 0.418 | 0.409 | 0.414 | 97.5 |
| | 19 | 0.429 | 0.421 | 0.424 | 0.425 | 100.1 |

TABLE 3E
Stability of UDCA solution according to Example IV at pH 9, 50 C.

| | Day | #1 | #2 | #3 | Average | Percentage |
|---|---|---|---|---|---|---|
| Ile | 0 | 0.291 | 0.286 | 0.282 | 0.286 | 100.0 |
| | 3 | 0.266 | 0.273 | 0.282 | 0.273 | 95.6 |
| | 6 | 0.277 | 0.274 | 0.272 | 0.274 | 95.9 |
| | 10 | 0.243 | 0.245 | 0.295 | 0.261 | 91.2 |
| | 13 | 0.246 | 0.269 | 0.236 | 0.250 | 87.4 |
| | 17 | 0.275 | 0.280 | 0.245 | 0.267 | 93.1 |
| Leu | 0 | 0.509 | 0.513 | 0.511 | 0.511 | 100.0 |
| | 3 | 0.485 | 0.487 | 0.492 | 0.488 | 95.5 |
| | 6 | 0.495 | 0.496 | 0.492 | 0.494 | 96.8 |
| | 10 | 0.470 | 0.467 | 0.528 | 0.488 | 95.6 |
| | 13 | 0.461 | 0.491 | 0.450 | 0.467 | 91.5 |
| | 17 | 0.468 | 0.516 | 0.500 | 0.495 | 96.9 |
| Val | 0 | 0.508 | 0.476 | 0.484 | 0.489 | 100.0 |
| | 3 | 0.463 | 0.487 | 0.485 | 0.478 | 97.8 |
| | 6 | 0.493 | 0.473 | 0.495 | 0.487 | 99.5 |
| | 10 | 0.441 | 0.428 | 0.471 | 0.447 | 91.3 |
| | 13 | 0.467 | 0.483 | 0.537 | 0.496 | 101.3 |
| | 17 | 0.499 | 0.495 | 0.501 | 0.498 | 101.8 |
| Sol | 0 | 0.341 | 0.316 | 0.328 | 0.328 | 100.0 |
| | 3 | 0.297 | 0.317 | 0.317 | 0.310 | 94.5 |
| | 6 | 0.313 | 0.291 | 0.314 | 0.306 | 93.2 |
| | 10 | 0.268 | 0.253 | 0.324 | 0.282 | 85.8 |
| | 13 | 0.270 | 0.266 | 0.334 | 0.290 | 88.3 |
| | 17 | 0.337 | 0.329 | 0.317 | 0.328 | 99.8 |
| UDCA | 0 | 0.389 | 0.385 | 0.389 | 0.388 | 100.0 |
| | 3 | 0.405 | 0.400 | 0.394 | 0.400 | 103.2 |
| | 6 | 0.427 | 0.411 | 0.416 | 0.418 | 107.9 |
| | 10 | 0.420 | 0.418 | 0.450 | 0.429 | 110.8 |
| | 13 | 0.465 | 0.434 | 0.441 | 0.447 | 115.3 |
| | 17 | 0.454 | 0.457 | 0.413 | 0.441 | 113.9 |

TABLE 3F
Stability of UDCA solution according to Example IV at pH 10, 50 C.

| | Day | #1 | #2 | #3 | Average | Percentage |
|---|---|---|---|---|---|---|
| Ile | 0 | 0.292 | 0.282 | 0.287 | 0.287 | 100.0 |
| | 2 | 0.253 | 0.237 | 0.239 | 0.243 | 84.7 |
| | 5 | 0.221 | 0.212 | 0.221 | 0.218 | 76.0 |
| | 7 | 0.219 | 0.215 | 0.207 | 0.214 | 74.5 |
| | 9 | 0.206 | 0.192 | 0.207 | 0.202 | 70.2 |
| Leu | 0 | 0.507 | 0.495 | 0.509 | 0.504 | 100.0 |
| | 2 | 0.462 | 0.442 | 0.442 | 0.449 | 89.1 |
| | 5 | 0.429 | 0.428 | 0.427 | 0.428 | 85.0 |
| | 7 | 0.410 | 0.417 | 0.414 | 0.414 | 82.1 |
| | 9 | 0.417 | 0.377 | 0.418 | 0.404 | 80.2 |
| Val | 0 | 0.480 | 0.506 | 0.471 | 0.486 | 100.0 |
| | 2 | 0.536 | 0.478 | 0.504 | 0.506 | 104.2 |
| | 5 | 0.371 | 0.445 | 0.400 | 0.405 | 83.5 |
| | 7 | 0.384 | 0.384 | 0.424 | 0.397 | 81.8 |
| | 9 | 0.389 | 0.354 | 0.362 | 0.368 | 75.8 |
| Sol | 0 | 0.368 | 0.376 | 0.331 | 0.358 | 100.0 |
| | 2 | 0.284 | 0.257 | 0.266 | 0.269 | 75.1 |
| | 5 | 0.053 | 0.217 | 0.192 | 0.154 | 43.0 |
| | 7 | 0.042 | 0.026 | 0.156 | 0.075 | 20.8 |
| | 9 | 0.033 | 0.019 | 0.023 | 0.025 | 7.0 |
| UDCA | 0 | 0.416 | 0.402 | 0.406 | 0.408 | 100.0 |
| | 2 | 0.402 | 0.397 | 0.400 | 0.399 | 97.9 |
| | 5 | 0.425 | 0.413 | 0.423 | 0.420 | 103.0 |
| | 7 | 0.406 | 0.402 | 0.408 | 0.406 | 99.4 |
| | 9 | 0.424 | 0.426 | 0.421 | 0.423 | 103.8 |

### Example IX

A ninth series of solution formulations that were prepared with soluble bile acids (as free form) and high molecular weight aqueous soluble starch conversion products according to the following guidelines did not show any precipitation at any pH within the selected, desired pH range. This formulation is modified based on the known analytical data for bear bile.

| | | |
|---|---|---|
| Soluble Bile Acid | 21 | g UDCA, |
| | 9 | g CA, and |
| | 9 | g CDCA |
| Starch Conversion Product | about 750 | g |
| Purified water to make | 1.0 | L |

An aqueous solution (400 mL) of soluble UDCA was prepared. Then the high molecular weight aqueous soluble starch conversion product was added to make a clear solution. Into the resulting clear solution, soluble CDCA, and soluble CA were added. The pH of this solution was adjusted by acid with high throughput sonication to prepare oral and topical dosage forms. Purified water or water for injection was added to make the total volume be 1.0 L.

### Example X

A tenth series of solution formulations that were prepared with soluble bile acids (as free form) and high molecular weight aqueous soluble starch conversion products according to the following guidelines did not show any precipitation at any pH within the selected, desired pH range. This formulation is modified based on the known analytical data for bear bile.

| | | |
|---|---|---|
| Soluble Bile Acid | 21 | g UDCA, |
| | 9 | g CA, and |
| | 9 | g CDCA |
| Starch Conversion Product | about 750 | g |
| Aqueous Soluble Ginseng Extract | 20 | g |
| Purified water to make | 1.0 | L |

An aqueous solution (400 mL) of soluble UDCA was prepared. Then the high molecular weight aqueous soluble starch conversion product was added to make a clear solution. Into the resulting clear solution, soluble CDCA, soluble CA, and aqueous soluble ginseng extract were added. The pH of this solution was adjusted by acid with high throughput sonication to prepare oral and topical dosage forms. Purified water or water for injection was added to make the total volume be 1.0 L.

### Example XI

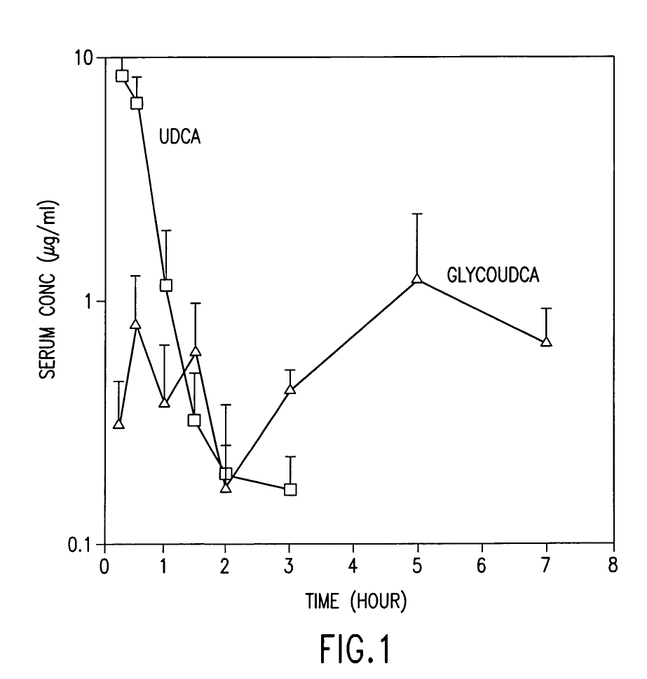

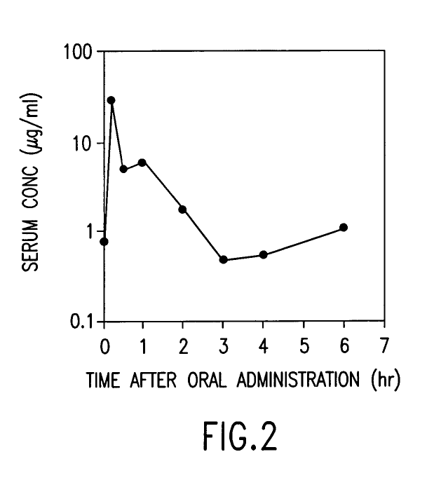

Aqueous solution formulations, according to this invention, containing 200 mg of ursodeoxycholic acid (UDCA), were prepared according to the method described in the above-described Example III and were administered to three healthy men having normal body weight after fasting. The hematic levels of UDCA and glyco UDCA were evaluated by means of well known analytical methods. After applying buffered serum to sep-pak column, methanol eluate was derivatized with phenacyl bromide at 80 C for 45 minutes. These phenacyl bromide derivatives were dissolved in acetonitrile in preparation for HPLC. The experimental results of the absorption measured at certain times after dosage administration include the total absorption expressed as the area under the serum concentration-time curve (AUC: μg/mL × hours), the maximum hematic concentration (Cₘₐₓ; μg/mL) that has been obtained, and the time (Tₘₐₓ; hour) in which said maximum concentration has been obtained. These results are reported in Table 4, FIG. 1, and FIG. 2.

The experimental pharmacokinetic tests of the aqueous solution formulations according to this invention carried out on men show substantial improvement in AUC, Cₘₐₓ and Tₘₐₓ in comparison with the best results from any dosage forms known presently. The maximum hematic concentration (Cₘₐₓ) in Table 4 shows an average of 8.43±1.69 μg/mL which is at least two times higher than that reported for use of enteric coated sodium salt of UDCA preparations and four times higher than that obtained using regular UDCA tablet preparations. Moreover, the time of peak concentration (Tₘₐₓ) which is related closely to the rate of absorption of UDCA from the aqueous solution formulations is 0.25 hours, at least three times faster than the fastest Tₘₐₓ previously known.

Table 4A and Table 4B show plasma concentration of UDCA and GUDCA measured in 3 men over time following on oral administration of the UDCA and GUDCA containing formulations according to Example VI and comparison of results against results of others employing different pharmaceutical formulations of UDCA.

Table 5 shows phamacokinetic parameters of UDCA in human after an oral administration of liquid formulation of UDCA. Cₘₐₓ is shown.

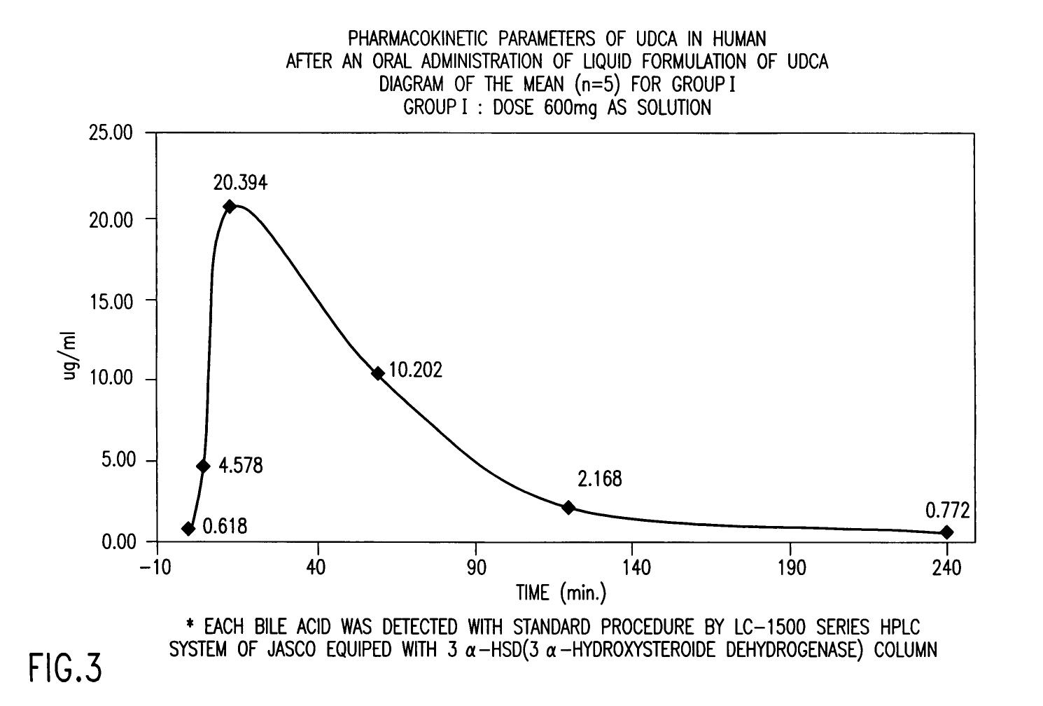

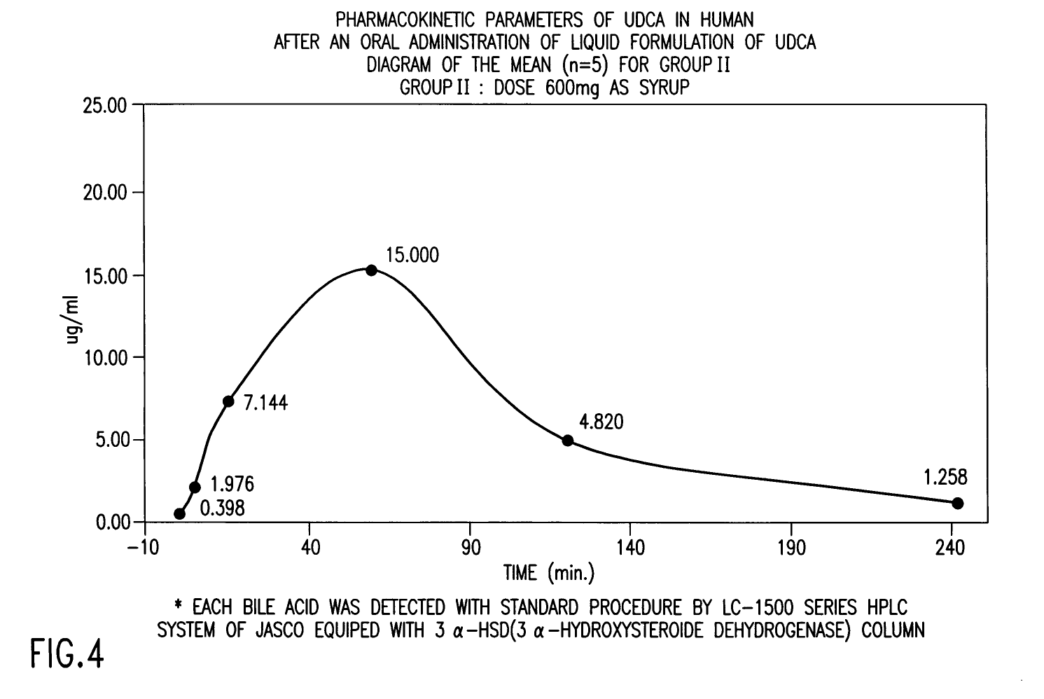

Taken together, the data in Tables 4 and 5 and FIGS. 3 and 4 illustrate the superiority of formulations of the instant invention over conventional formulations with respect to Cₘₐₓ and Tₘₐₓ the instant The inventive solutions were effect without any break-down of the solution system caused by the pH of the environment in the stomach and intestines. The therapeutic potential of bile acid and possibly even added pharmaceuticals may be more fully realized using the formulations of the invention. When the therapeutically active ingredients in aqueous solution forms are not precipitated as solid by acidic gastric juices in the stomach and by the various alkaline pH levels of the intestine, the formulation overcomes as a natural consequence, the scarce bioavailability resulted by the unexpected, undesirable results for the extent and the rate of release by disintegration, dissolution and/or diffusion should be overcome.

TABLE 4A
Plasma concentration of UDCA and GUDCA after an oral administration of
this invention at a dose of 200 mg to three men

| UDCA | | | | | GUDCA | | | |
|---|---|---|---|---|---|---|---|---|
| Time(h) | #1 | #2 | #3 | mean | #1 | #2 | #3 | mean |
| 0.25 | 5.1202 | 10.9171 | 9.159 | 8.43 ± 1.6 | 0.1419 | 0.4549 | 0.3328 | 0.31 ± 0.0 |
| 0.5 | 4.4528 | 7.7432 | 7.4395 | 6.55 ± 1.0 | 0.2564 | 1.2455 | 0.864 | 0.79 ± 0.2 |
| 1 | 1.6921 | 1.546 | 0.2163 | 1.15 ± 0.4 | 0.2162 | 0.6926 | 0.2142 | 0.37 ± 0.1 |
| 1.5 | 0.5256 | 0.2759 | 0.168 | 0.32 ± 0.1 | 1.1573 | 0.1929 | 0.4752 | 0.61 ± 0.2 |
| 2 | 0.2349 | 0.2176 | 0.1227 | 0.19 ± 0.0 | 0.4013 | 0.0312 | 0.0657 | 0.17 ± 0.1 |
| 3 | 0.1237 | N.D. | 0.2074 | 0.17 ± 0.0 | 0.5085 | 0.4303 | 0.3315 | 0.42 ± 0.0 |
| 5 | | | | | 1.9205 | 0.0229 | 1.6311 | 1.18 ± 0.6 |
| 7 | | | | | 0.5328 | 0.4797 | 0.91 | 0.64 ± 0.1 |
| AUC (μg | 4.32 | 6.6 | 5.47 | 5.46 ± 0.6 | 6.26 | 2.22 | 4.65 | 4.38 ± 1.1 |
| Cₘₐₓ (μg/mL) | 5.21 | 10.92 | 9.16 | 8.43 ± 1.6 | 1.92 | 1.25 | 1.63 | 1.6 |
| Tₘₐₓ(h) | 0.25 | 0.25 | 0.25 | 0.25 | 5 | 0.5 | 5 | 3.5 ± 1.5 |

TABLE 4B
Pharmacokinetic parameters of UDCA in human after an oral
administration of UDCA (M ± S.E.)

| | Cₘₐₓ (μg/mL) | Tₘₐₓ (hr) |
|---|---|---|
| Roda et al. (1994) | | |
| UDCA gelatine capsule, 450 mg | 2.59 | 3.8 |
| NaUDC gelatine capsule, 475 mg | 3.42 | 2.4 |
| NaUDC enteric-coated, 475 mg | 10 | 3.4 |
| Nagamatsu et al. (1997) | | |
| UDCA 200 mg | 1.9 ± 0.25 | 1.5 ± 0.4 |
| UDCA 400 mg | 7.09 ± 1.43 | 0.8 ± 0.2 |
| UDCA in this invention, 200 mg | 8.43 ± 1.69 | 0.25 |

TABLE 5A
Pharmacokinetic parameter (Cₘₐₓ) of UDCA in human after oral administration
of a liquid solution containing 600 mg UDCA per day.

| Time (min) | Person #1 | Person #2 | Person #3 | Person #4 | Person #5 | Average | Std. Dev. |
|---|---|---|---|---|---|---|---|
| 0 | 0.35 | 1.63 | 0.40 | 0.00 | 0.71 | 0.618 | 0.619 |
| 5 | 2.51 | 9.79 | 1.68 | 2.65 | 6.26 | 4.578 | 3.405 |
| 15 | 12.50 | 47.46 | 8.34 | 11.84 | 21.83 | 20.394 | 15.933 |
| 60 | 9.72 | 6.46 | 7.77 | 9.81 | 17.25 | 10.202 | 4.183 |
| 120 | 3.77 | 1.71 | 1.40 | 1.15 | 2.81 | 2.168 | 1.097 |
| 240 | 0.65 | 0.93 | 0.50 | 0.48 | 1.30 | 0.772 | 0.346 |

TABLE 5B
Pharmacokinetic parameter (Cₘₐₓ) of UDCA in human after an oral
administration of a syrup containing 600 mg UDCA per day.

| Time (min) | Person #1 | Person #2 | Person #3 | Person #4 | Person #5 | Average | Std. Dev. |
|---|---|---|---|---|---|---|---|
| 0 | 0.62 | 0.58 | 0.38 | 0.00 | 0.41 | 0.398 | 0.246 |
| 5 | 2.76 | 2.63 | 0.83 | 1.42 | 2.24 | 1.976 | 0.827 |
| 15 | 7.80 | 4.45 | 3.54 | 5.85 | 14.08 | 7.144 | 4.197 |
| 60 | 16.08 | 20.33 | 8.76 | 12.06 | 17.77 | 15.000 | 4.605 |
| 120 | 3.98 | 4.24 | 5.09 | 7.79 | 3.00 | 4.820 | 1.820 |
| 240 | 0.81 | 0.99 | 1.47 | 1.85 | 1.17 | 1.258 | 0.411 |

### Example XII

An eleventh series of solution formulations that were prepared with soluble bile acids (as free acid), high molecular weight aqueous soluble starch conversion products, and non-starch polysaccharides (dried powder of liquid glucose, e.g. commercial corn syrup solid) according to the following guidelines did not show any precipitation at any pH within the selected, desired range of pH values.

| | |
|---|---|
| Soluble Bile Acid | 200 mg UDCA (10 mg to 3 g) |
| Dried Powder of Liquid Glucose | 25 g (0.25 g to 75 g) |
| Soluble Non-Starch Polysaccharide | 25 g (0.25 g to 75 g) |
| Purified water to make | 100 mL |

An aqueous solution (45 mL) of soluble UDCA was prepared. Then the high molecular weight aqueous soluble starch conversion product (i.e. dried liquid glucose), which has DE of 5-40, was added to make a clear solution. The pH of this solution was adjusted by acid with high throughput sonication to prepare oral and topical dosage forms. Purified water or water for injection was added to make the total volume be 1.0 L.

Into the resulting clear solution, a soluble non starch polysaccharide (guar gum, pectin, etc.) was added into pH-adjusted clear solution with agitation. Purified water was added to make the total volume to 100 mL.

### Example XIII

### Mixture Solution

The formulations of Examples VIII, IX, and X include bismuth compound. In each of these examples, solution formulations were prepared by adding an amount of an ammonium salt of bismuth sulfate sufficient to provide the indicated amount of bismuth hydroxide.

A twelfth series of solution formulations that were prepared with soluble bile acids (as free acid), high molecular weight aqueous soluble starch conversion products, and bismuth compounds according to the following guidelines did not show any precipitation at any pH within the selected, desired range of pH values.

| | |
|---|---|
| Soluble Bile Acid | 20 g UDCA |
| Bismuth Citrate | 5 g |
| Corn Syrup Solid | 500 g |
| Citric Acid | q.s. |
| Purified water to make | 1.0 L |

A 3 mL aliquot of IN NaOH was poured into water (200 mL) followed by addition of UDCA. The bismuth citrate was added to the resulting clear solution, while maintaining pH 9-10, along with 200 mL of water. Next, the corn syrup solid, as one high molecular weight aqueous soluble starch conversion product, was added portion by portion to the resulting clear solution and dissolved with agitation to make a clear solution. The pH of this resulting clear solution was adjusted (to pH 3 to pH 5) by citric acid with high throughput sonication, which may accelerate solubilization of the bismuth compound. Purified water was added to adjust the total volume to 1.0 L.

### Example XIV

### UDCA-Thick Syrup (30 g UDCA/L)

A thirteenth series of solution formulations that were prepared according to the following guidelines did not show any precipitation at any pH within the selected, desired range of pH values.

| | |
|---|---|
| Soluble Bile Acid | 30 g UDCA |
| 1 N NaOH | 4 mL |
| Maltodextrin | 750 g |
| Citric Acid or Lactic Acid | q.s. |
| Purified water to make | 1.0 L |

The UDCA is first dissolved in NaOH solution and then diluted with 250 mL of water. Next, the maltodextrin, as one high molecular weight aqueous soluble starch conversion product that has a lower DE, was added portion by portion with vigorous agitation. The pH of this resulting clear solution was adjusted (to pH 3) by addition of citric acid with high throughput sonication. Purified water was added to adjust the total volume to 1.0 L.

### Example XV

### UDCA-Paste (45 g UDCA/L)

A fourteenth series of solution formulations that were prepared according to the following guidelines did not show any precipitation at any pH within the selected, desired range of pH values.

| | |
|---|---|
| Soluble Bile Acid | 45 g UDCA |
| 1 N NaOH | 135 mL |
| Maltodextrin | 1,575 g |
| Citric Acid or Lactic Acid | q.s. |
| Purified water to make | 1.0 L |

The UDCA is first dissolved in 135 mL of a 1N NaOH solution. Next, to the resulting clear solution were added the bismuth citrate and 200 mL of water. Then, 1,575 g of maltodextrin was added portion by portion with vigorous agitation. The resulting solution was titrated to pH 3 by the addition of citric acid. Purified water was added to adjust the total volume to 1.0 L.

Five human subjects were provided with dosage forms prepared according to this Example. The results are shown in Tables 5A and 5B and rendered graphically in FIGS. 3 and 4. A comparison of the sharp peak of FIG. 3 with the broad peak of FIG. 4 indicates that, by adjusting the dosage form, a practitioner may manipulate the bile acid Cₘₐₓ and Tₘₐₓ.

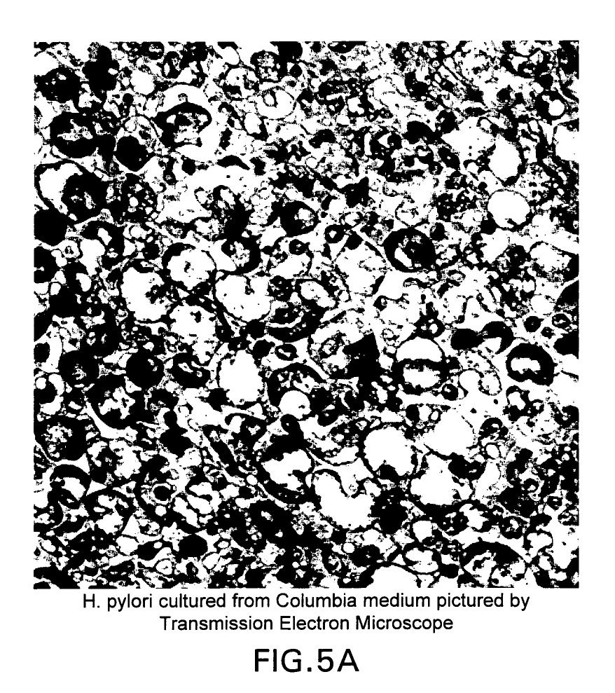

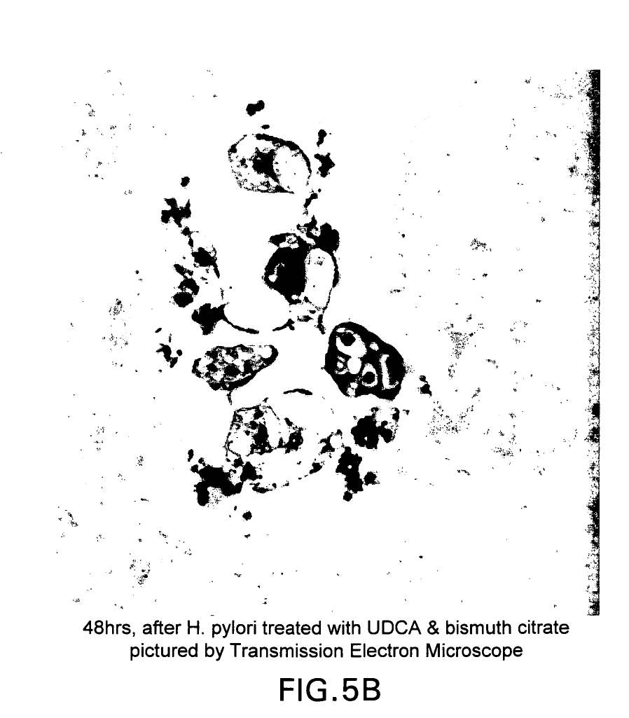

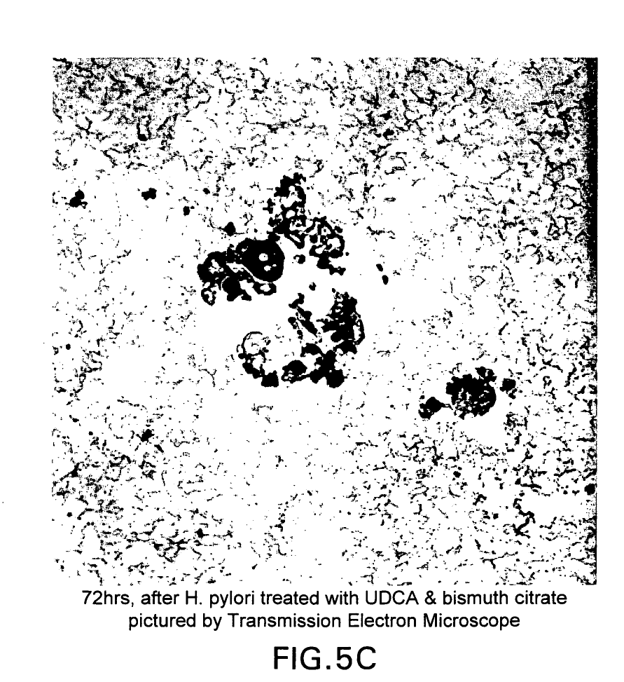

H. pylori were cultured on Columbia Blood Agar Base (CRAB) media containing a preparation of Example IX. 2 L of CRAB plates were prepared which contained 9.9 g of CRAB, 9.1 g of tryptic soy agar, 50 mL of sheep blood, vacomycin, amphotericin B, polymixin B, 2 mL of Example IX, and 358 mL distilled water. After 48 or 72 hours of microaerophillic incubation, bacteria were fixed using Kamovsky's fixative and embedded in epon. Electron micrographs of H. pylori cells are shown in FIGS. 5A to 5C.

### Example XVI

### UDCA-Paste (45 g UDCA/L)

A fifteenth series of solution formulations that were prepared according to the following guidelines did not show any precipitation at any pH within the selected, desired range of pH values.

| | |
|---|---|
| Soluble Bile Acid | 45 g UDCA |
| 1 N NaOH | 135 mL |
| Corn syrup solid | 2,300 g |
| Citric acid or lactic acid | 50 g |
| Purified water to make | 1.0 L |

The UDCA is first dissolved in 135 mL of a IN NaOH solution. Next, to the resulting clear solution were added the bismuth citrate and 150 mL of water. Then, 2,300 g of corn syrup solid was added portion by portion with vigorous agitation. The resulting solution was titrated to pH 3 by the addition of citric acid. Purified water was added to adjust the total volume to 1.0 L.

### Example XVII

### Mixture Solution of UDCA (22 G) and CDCA (3 G)

A sixteenth series of solution formulations that were prepared according to the following guidelines did not show any precipitation at any pH within the selected, desired range of pH values.

| | |
|---|---|
| UDCA | 22 g |
| 1 N NaOH | 75 mL |
| CDCA | 3 g |
| Maltodextrin | 875 g |
| Bismuth citrate | 4 g |
| Citric acid or lactic acid | q.s. |
| Purified water to make | 1.0 L |

The UDCA and CDCA are first dissolved in 75 mL of a 1N NaOH solution. Next, to the resulting clear solution were added the bismuth citrate and 240 mL of water. Then, 875 g of maltodextrin was added portion by portion with vigorous agitation. The resulting solution was titrated to pH 3 by the addition of citric acid. Purified water was added to adjust the total volume to 1.0 L.

### Example XVIII

### Mixture Solution of UDCA (22 G) and CDCA (3 G)

A seventeenth series of solution formulations that were prepared according to the following guidelines did not show any precipitation at any pH within the selected, desired range of pH values.

| | |
|---|---|
| UDCA | 22 g |
| 1 N NaOH | 75 mL |
| CDCA | 3 g |
| Corn syrup solid | 1,320 g |
| Bismuth citrate | 4 g |
| Citric acid or lactic acid | q.s. |
| Purified water to make | 1.0 L |

The UDCA and CDCA are first dissolved in 75 mL of a 1N NaOH solution. Next, to the resulting clear solution were added the bismuth citrate and 240 mL of water. Then, 1,320 g of corn syrup solid was added portion by portion with vigorous agitation. The resulting solution was titrated to pH 3 by the addition of citric acid. Purified water was added to adjust the total volume to 1.0 L.

### Example XIX

The effect of treating H. pylori infected mice with a solution dosage form of the invention was tested. Six week old C57BL/6 female mice were infected by feeding a diet comprising 10⁹ CFU/mL H. pylori, SS1 strain. The animals consumed this feed twice, one week apart. Subsequently, 0.2 mL of a solution dosage form according to Example XIII was administered to four infected animals once per day for one week. Two animals were sacrificed one week following administration of the last dose of the inventive solution. The remaining two animals were sacrificed four weeks following administration of the last dose of the inventive solution. Whole stomachs were washed with saline to remove mucosa and debris. A sample of stomach tissue from each animal was subjected to a CLO test using a rapid urease test kit (Delta West, Australia). Each residual stomach was fixed with 10% formalin solution and embedded with paraffin. Sections (4 μm thick) were collected on glass slides and stained with H&E staining solution and Warthin staining solution. Tissue was evaluated for pathological status by conventional light microscopy.

The results, summarized in Table 6, indicate that the urease test results were negative for mice passed one week after discontinuing administration of the liquid dosage form, and H. pylori was not seen in Warthin examination. Of the other two mice, one showed a negative urease test and no H. pylori were seen by Warthin examination. The other, however, yielded a positive urease test although only a few H. pylori were seen in Warthin examination.

TABLE 6

| Weeks After | | | Warthin |
|---|---|---|---|
| Treatment | Animal | Urease Test | Examination |
| 1 | 1 | Negative | No H. pylori |
| 1 | 2 | Negative | No H. pylori |
| 4 | 3 | Negative | No H. pylori |
| 4 | 4 | Positive | A few H. pylori |

### Example XX

Assays for growth of H. pylori on media containing UDCA, bismuth citrate or both UDCA and bismuth citrate were performed. For these assays the following media was used:
000112B-1 having a pH of 4.0 and comprising 525 g/L maltodextrin and 15 g/L UDCA.
OSABY having a pH of 3.7 and comprising 1 kg/L corn syrup solid and 6 g/L bismuth citrate.

Three assays were performed to assess the growth capacity of H. pylori in the presence of UDCA, bismuth or both wherein the pH, concentration, and length of exposure was varied.

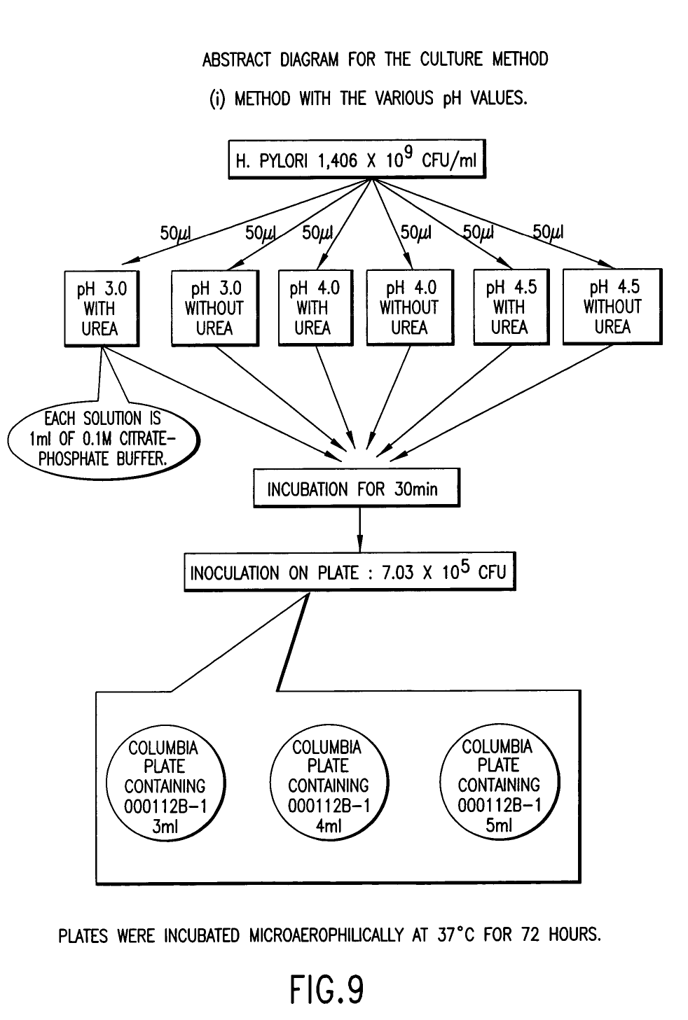

In the first, Helicobacter pylori was suspended in physiological saline to give about 10⁹ organisms per milliliter. 50 μL of this inoculum was transferred to tubes containing 1 mL of citrate-phosphate buffer at pH 3.0, 4.0, and 4.5. Paired tubes were prepared with and without 6 mM Urea. Following a 30 minute room temperature incubation, the suspensions were subcultured on agar plates containing 000112B-1 using a 1 μL loop. Plates were incubated microaerophilically at 37 C for 72 hours. This procedure is illustrated in FIG. 9.

As shown in Table 7, H. pylori grew poorly on pH 3 and pH 4 control media. Table 7 further shows that H. pylori does not grow on pH 3 and pH 4 media containing UDCA. The designations “3 ml”, “4 ml” and “5 ml” refer to the total volume of 000112B-1 media per plate. “PBS” is phosphate buffered saline at pH 7.0.

TABLE 7

| | | | Urease Test | | | |
|---|---|---|---|---|---|---|
| Plate | pH | Urea | 1-2 sec. | 10 min. | 2 hr. | 20 hr. |
| Control | 3.0 | Yes | FO | FO | O | O |
| | | No | FO | FO | O | O |
| | 4.0 | Yes | FO | FO | O | O |
| | | No | FO | FO | O | P |
| | 4.5 | Yes | FP | FP | P | P |
| | | No | FO | O | FP | P |
| | PBS | Yes | O | FP | P | P |
| | | No | O | FP | P | P |
| 000112B-1 | 3.0 | Yes | Y | Y | Y | Y |
| (3 mL) | | No | Y | Y | Y | Y |
| | 4.0 | Yes | Y | Y | FO | O |
| | | No | Y | Y | Y | FO |
| | 4.5 | Yes | FP | FP | P | P |
| | | No | FP | FP | P | P |
| 000112B-1 | 3.0 | Yes | Y | Y | Y | Y |
| (4 mL) | | No | Y | Y | Y | Y |
| | 4.0 | Yes | Y | Y | Y | FO |
| | | No | Y | Y | FO | FO |
| | 4.5 | Yes | FP | FP | P | P |
| | | No | FP | FP | FP | P |
| 000112B-1 | 3.0 | Yes | Y | Y | Y | Y |
| (5 mL) | | No | Y | Y | Y | Y |
| | 4.0 | Yes | Y | Y | FO | FO |
| | | No | Y | Y | Y | Y |
| | 4.5 | Yes | FP | FP | P | P |
| | | No | FP | FP | P | P |

Key

| | Y | FO | O | FP | P |
|---|---|---|---|---|---|
| Color | Yellow | Faint Orange | Orange | Faint Pink | Pink |
| Helicobacter | None | Very Rare | Rare | Exist | Many |

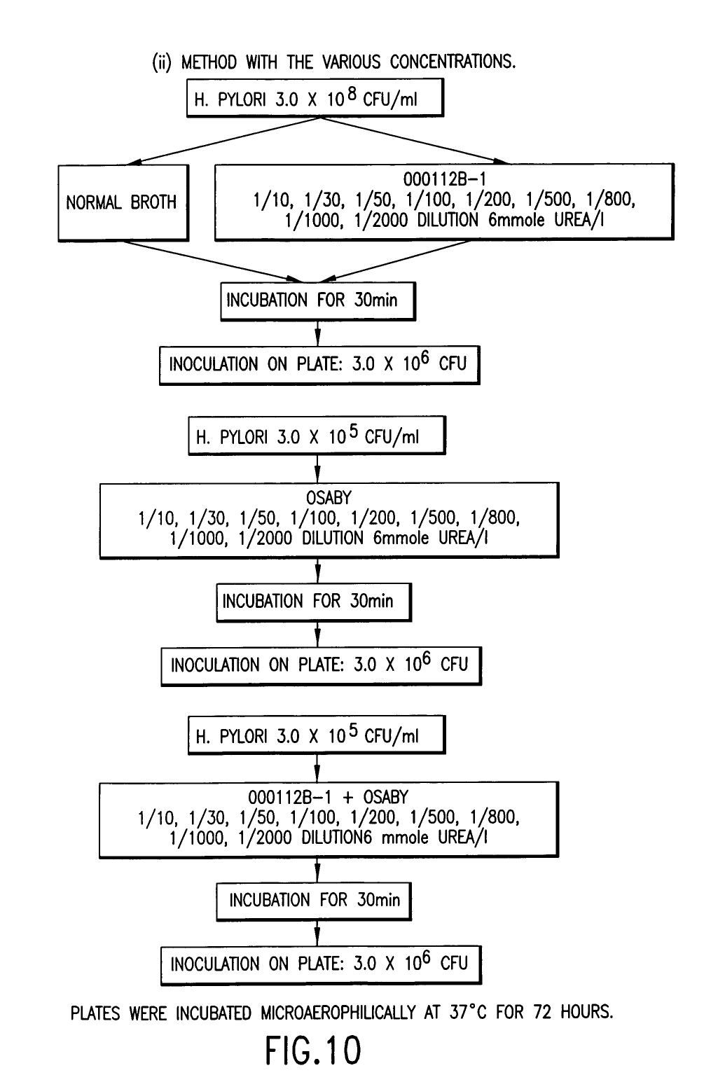

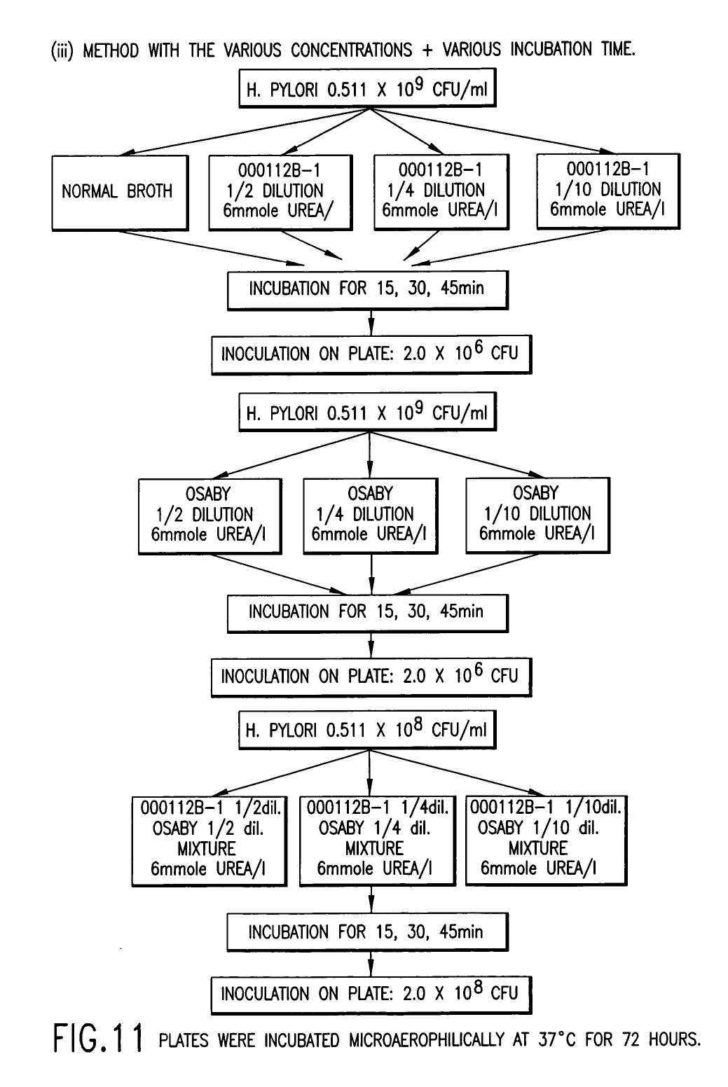

In the second assay, Helicobacter pylori was suspended in physiological saline to give about 10⁹ organisms per milliliter. 50 μL of this inoculum was transferred to tubes containing 1 mL of citrate-phosphate buffer at various concentrations of plating media such as 1/10, 1/30, 1/50, 1/100, 1/200, 1/500, 1/800, 1/1000, 1/2000. All tubes were prepared with 6 mM Urea. Following a 30 minute room temperature incubation, the suspensions were subcultured on agar plates using a 1 μL loop. These plates were substantially free of bismuth and bile acids. Plates were incubated microaerophilically at 37 C for 72 hours. This procedure is illustrated in FIG. 10.

Table 8 shows urease test results following 72 hours of growth of H. pylori on media prepared with dilutions of UDCA (000112B-1) bismuth citrate (OSABY) or both UDCA and bismuth citrate. Poor growth of H. pylori on media containing either UDCA or bismuth citrate was observed (Table 8). Growth of H. pylori was further attenuated when cultured on media containing both UDCA and bismuth citrate (Table 8).

TABLE 8

| | Urease Test | | | |
|---|---|---|---|---|
| Plate | Immediately | 10 min. | 30 min. | 60 min. |
| 000112B-1 | | | | |
| Control | P | P | P | P |
| 1/10 | Y | FP | P | P |
| 1/30 | Y | FP | P | P |
| 1/50 | Y | FP | P | P |
| 1/100 | Y | FP | P | P |
| 1/200 | Y | FP | P | P |
| 1/500 | Y | FP | P | P |
| 1/800 | FP | P | P | P |
| 1/1000 | FP | P | P | P |
| 1/2000 | FP | P | P | P |
| OSABY | | | | |
| Control | P | P | P | P |
| 1/10 | Y | FP | P | P |
| 1/30 | Y | FP | P | P |
| 1/50 | Y | FP | P | P |
| 1/100 | FP | FP | P | P |
| 1/200 | FP | FP | P | P |
| 1/500 | FP | FP | P | P |
| 1/800 | FP | P | P | P |
| 1/1000 | FP | P | P | P |
| 1/2000 | FP | P | P | P |
| 000122B-1 + OSABY | | | | |
| Control | P | P | P | P |
| 1/10 | Y | FP | FP | FP |
| 1/50 | Y | Y | Y | Y |
| 1/100 | Y | Y | Y | Y |
| 1/500 | Y | FP | FP | FP |
| 1/1000 | Y | FP | P | P |

Key

| | Y | FO | O | FP | P |
|---|---|---|---|---|---|
| Color | Yellow | Faint Orange | Orange | Faint Pink | Pink |
| Helicobacter | None | Very Rare | Rare | Exist | Many |

In the third assay, Helicobacter pylori was suspended in physiological saline to give about 10⁹ organisms per milliliter. 50 μL of this inoculum was transferred to tubes containing 1 mL of citrate-phosphate buffer at various concentrations such as ½, ¼, and 1/10 for 15 minutes, ½, ¼, and 1/10 for 30 minutes, and ½, ¼, and 1/10 for 45 minutes. Paired tubes were inoculated with and without 6 mM Urea. Following a 30 minute room temperature incubation, the suspensions were subcultured on agar plates using a 1 μL loop. These plates were substantially free of bismuth and bile acids. Plates were incubated microaerophilically at 37 C for 72 hours. This procedure is illustrated in FIG. 11.

Table 9 shows urease test results following 72 hours of growth of H. pylori on media prepared with dilutions of UDCA (000112B-1) bismuth citrate (OSABY) or both UDCA and bismuth citrate. As indicated, longer exposure times increased the adverse effect of the solutions on H. pylori.

TABLE 9

| | Incubation | Urease Test (min.) | | | | |
|---|---|---|---|---|---|---|
| Dilution | Time (min.) | 1 | 30 | 60 | 120 | 240 |
| 000112B-1 | | | | | | |
| Control | | P | P | P | P | P |
| ½ | 15 | Y | Y | Y | Y | Y |
| | 30 | Y | Y | Y | Y | Y |
| | 45 | Y | Y | Y | Y | Y |
| ¼ | 15 | Y | FO | FP | P | P |
| | 30 | Y | Y | FO | FO | FO |
| | 45 | Y | Y | Y | Y | Y |
| 1/10 | 15 | Y | FO | FO | FO | FO |
| | 30 | Y | FO | FO | O | P |
| | 45 | Y | Y | Y | FO | FO |
| OSABY | | | | | | |
| Control | | P | P | P | P | P |
| ½ | 15 | Y | Y | Y | Y | Y |
| | 30 | Y | Y | Y | Y | Y |
| | 45 | Y | Y | Y | Y | Y |
| ¼ | 15 | Y | Y | Y | Y | Y |
| | 30 | Y | Y | Y | Y | Y |
| | 45 | Y | Y | Y | Y | Y |
| 1/10 | 15 | Y | FO | FO | FO | P |
| | 30 | Y | Y | Y | Y | Y |
| | 45 | Y | Y | Y | Y | Y |
| 000122B-1 + OSABY | | | | | | |
| Control | | P | P | P | P | P |
| ½ | 15 | Y | Y | Y | Y | Y |
| | 30 | Y | Y | Y | Y | Y |
| | 45 | Y | Y | Y | Y | Y |
| ¼ | 15 | Y | Y | Y | Y | Y |
| | 30 | Y | Y | Y | Y | Y |
| | 45 | Y | Y | Y | Y | Y |
| 1/10 | 15 | Y | Y | Y | FO | FO |
| | 30 | Y | FO | FO | FO | P |
| | 45 | Y | Y | Y | Y | Y |

### Example XXI

In the some embodiments of the examples I to XX, dried form may be prepared by the evaporation under vacuum. Solution formulations of bile acid compositions were dried in the rotary evaporator at 90-95° C. under the vacuum 1.3×10⁻¹ Pa.

In come embodiments of the examples I to XX, spray-dried dried form may be prepared in the spray-drying equipped with a centrifugal atomizer under the following conditions; the feed liquid used in this system is approximate 30-40% solution of an aqueous soluble starch conversion product and feed flow rate is 50-70 mL/min, the inlet temperature is 150-180° C. and the outlet temperature is 50-100° C. The feed liquid was atomized by a centrifugal atomizer which is 30,000 rpm as rotational speed.

In the some embodiment of the examples I to XX, the granules derived from the solution formulations of bile acid compositions were produced in the fluid bed. The dried powder of the solution formulations of bile acid compositions (20 kg, 100-200 mesh) and the corn starch (9 kg) were placed in the fluid bed and were mixed by using air. Afterwards the binder solution (700 g of hydroxypropylmethyl cellulose in 22 L of water) was sprayed on the fluidizing powder bed using a peristaltic pump. The spraying process was carried out according to the settings of the process variables for the specific run. During the spraying process, every 10 min, ±10 g samples were taken from the powder bed for moisture content determination by the loss on drying. Spraying was continued until all the binder solution was used. The wetted granules were dried by fluidizing them with an inlet air temperature of 75° C. The drying cycle was terminated when an outlet air temperature of 35° C. was reached, indicating that the granules were dried sufficiently.

In the some embodiment of the examples I to XX, the enteric coated granules derived from the solution formulations of bile acid compositions were produced in the top-spray granulator, bottom-spray granulator or tangential-spray granulator. Dissolved the ethylcellulose N 100 (1.6 kg) in anhydrous ethanol and spray this solution and any additional ethanol into the fluidized dry powder of bile acid compositions (9 kg). Cease spraying when good granules are produced. Dry to approximately 3% moisture.

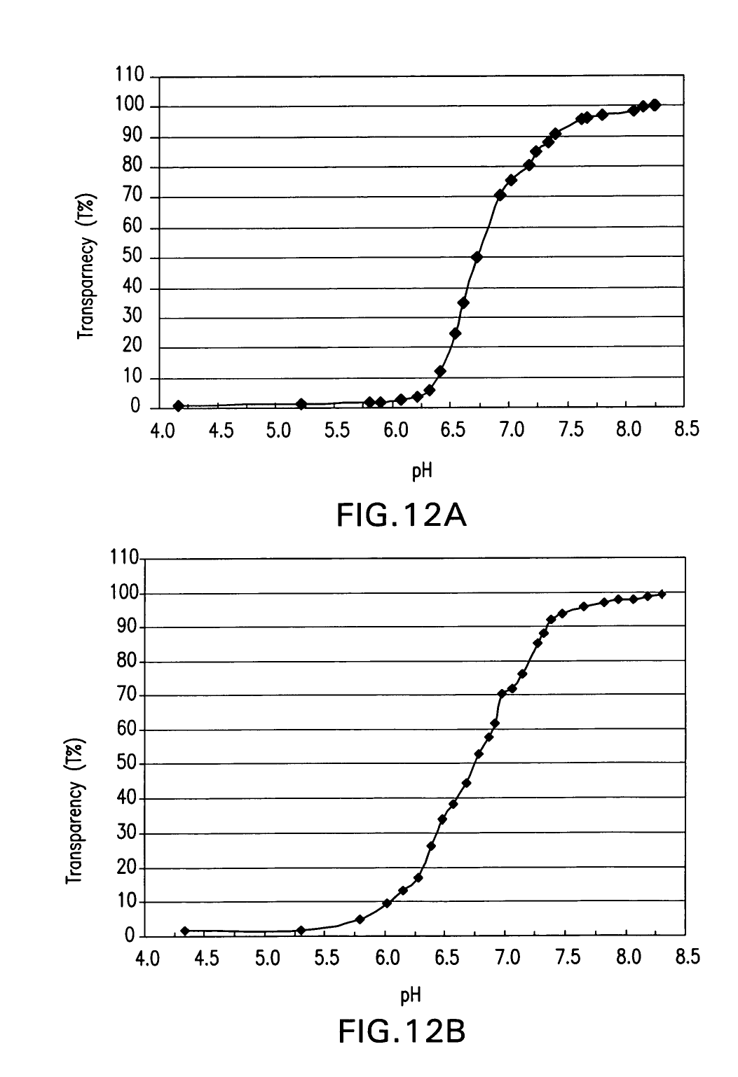

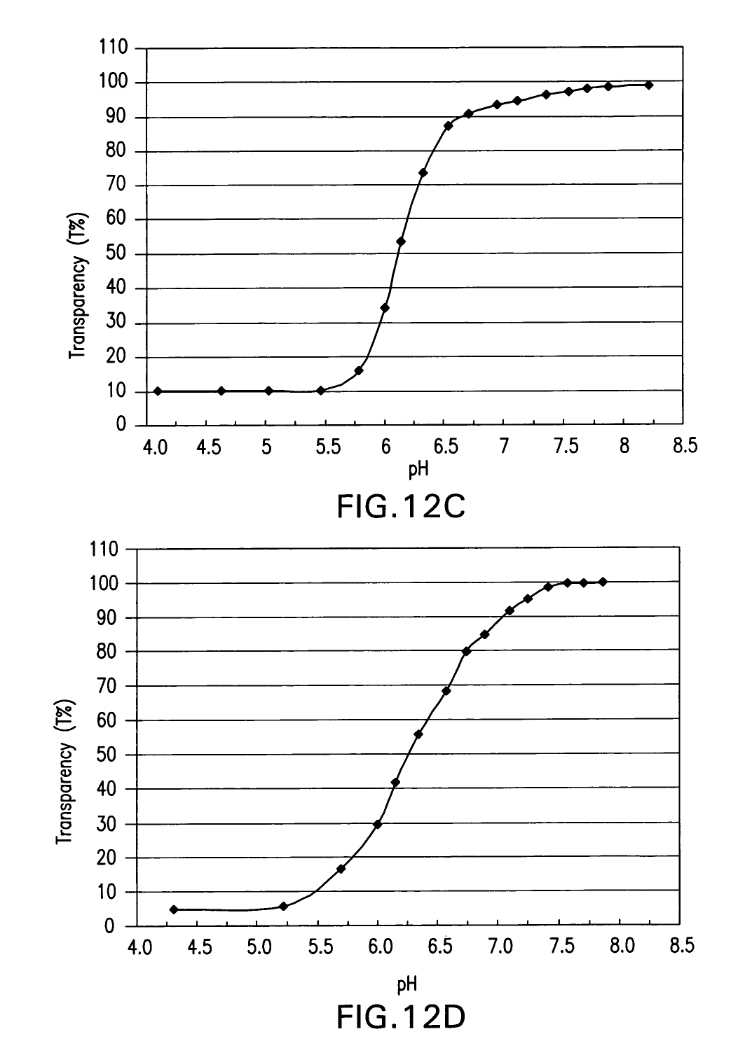

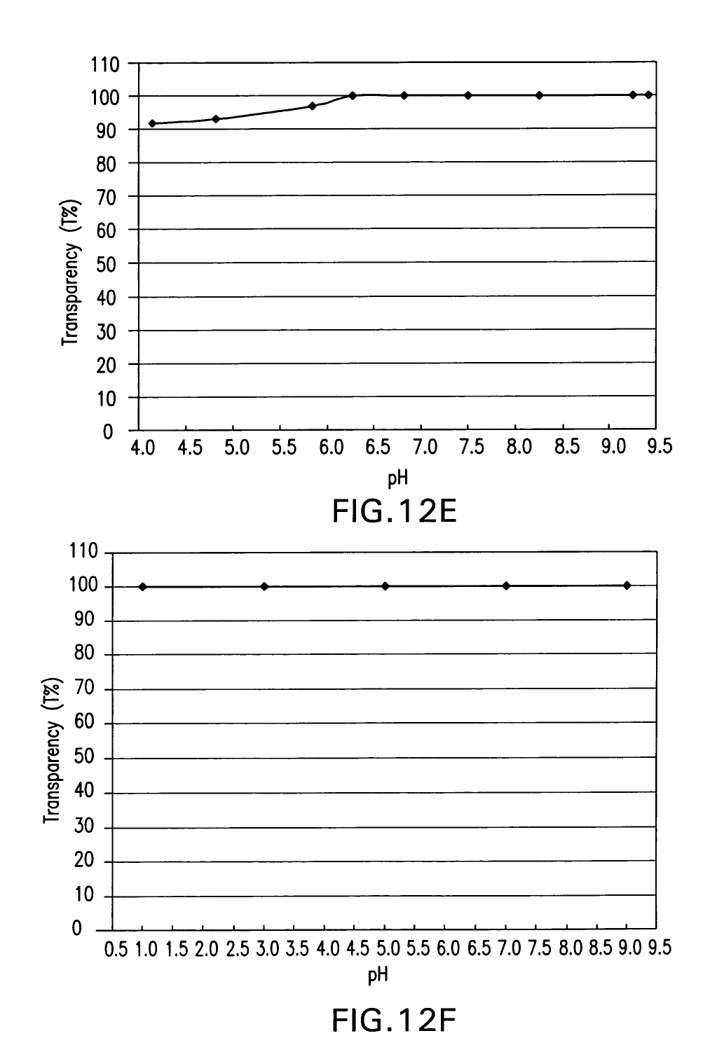

ASSAY. (Table 10 and FIG. 12). Increased amounts of maltodextrin (DE=15) as an aqueous soluble starch conversion product in the primary solution are associated with increased solubility of the dried material at low pH in the secondary solution, particularly within 2 minute. These results indicate that maltodextrin is excellent redissolving agent for the dried form of a primary aqueous solubilized bile acid formulation.

TABLE 10
Amount of maltodextrin in the Solution

| 4 g | | 5 g | | 6 g | | 7 g | | 8 g | | 9 g | |
|---|---|---|---|---|---|---|---|---|---|---|---|
| pH | T % | pH | T % | pH | T % | pH | T % | pH | T % | pH | T % |
| 4.16 | 0.88 | 4.34 | 1.67 | 4.09 | 10.18 | 4.31 | 4.79 | 4.15 | 91.82 | 1 | 100 |
| 5.22 | 1.56 | 5.3 | 1.77 | 4.626 | 10.16 | 5.223 | 5.79 | 4.81 | 93.03 | 3 | 100 |
| 5.8 | 1.87 | 5.79 | 4.83 | 5.026 | 10.18 | 5.69 | 16.71 | 5.84 | 96.9 | 5 | 100 |
| 5.9 | 1.92 | 6.02 | 9.52 | 5.46 | 10.21 | 6 | 29.44 | 6.27 | 100 | 7 | 100 |
| 6.08 | 2.6 | 6.15 | 13.36 | 5.78 | 16.06 | 6.15 | 41.59 | 6.82 | 100 | 9 | 100 |
| 6.21 | 3.77 | 6.28 | 17.08 | 6 | 34.23 | 6.342 | 55.76 | 7.5 | 100 | | |
| 6.32 | 5.96 | 6.39 | 26.22 | 6.14 | 53.25 | 6.57 | 68.36 | 8.25 | 100 | | |
| 6.41 | 12.27 | 6.48 | 33.99 | 6.33 | 73.6 | 6.74 | 79.95 | 9.25 | 100 | | |
| 6.54 | 24.76 | 6.57 | 38.26 | 6.54 | 87.16 | 6.89 | 84.66 | 9.42 | 100 | | |
| 6.61 | 34.99 | 6.68 | 44.34 | 6.71 | 90.61 | 7.1 | 91.55 | | | | |
| 6.73 | 49.89 | 6.78 | 52.65 | 6.948 | 93.5 | 7.25 | 95.1 | | | | |
| 6.93 | 70.66 | 6.87 | 57.73 | 7.116 | 94.67 | 7.41 | 98.48 | | | | |
| 7.02 | 75.67 | 6.92 | 61.67 | 7.35 | 96.24 | 7.57 | 99.65 | | | | |
| 7.18 | 80.5 | 6.98 | 70.2 | 7.54 | 97.11 | 7.7 | 99.67 | | | | |
| 7.24 | 85 | 7.06 | 71.88 | 7.69 | 98.15 | 7.86 | 100 | | | | |
| 7.34 | 88 | 7.145 | 75.99 | 7.87 | 98.54 | | | | | | |
| 7.4 | 90.9 | 7.27 | 85.01 | 8.21 | 99 | | | | | | |
| 7.62 | 95.54 | 7.32 | 87.88 | | | | | | | | |
| 7.665 | 96.27 | 7.38 | 92.17 | | | | | | | | |
| 7.8 | 97.15 | 7.47 | 93.67 | | | | | | | | |
| 8.07 | 98.46 | 7.65 | 95.79 | | | | | | | | |
| 8.15 | 99.67 | 7.82 | 97.05 | | | | | | | | |
| 8.24 | 100 | 7.94 | 97.75 | | | | | | | | |
| 8.25 | 100 | 8.06 | 97.9 | | | | | | | | |
| | | 8.18 | 98.75 | | | | | | | | |

1. A dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and combinations thereof; and
(b) an aqueous soluble starch conversion product;
wherein the first material and the aqueous soluble starch conversion product both remain in solution for all pH values of the solution within a selected range of pH values.

2. A dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and combinations thereof; and
(b) an aqueous soluble starch conversion product having a Dextrose Equivalency of from about 5 to about 10;
wherein the first material and the aqueous soluble starch conversion product both remain in solution for all pH values within the range of pH 6.5 to pH 8.

3. A dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and combinations thereof;
(b) a second material consisting of an aqueous soluble starch conversion product; and;
(c) a third material selected from the group consisting of a resistant maltodextrin and an aqueous soluble non-starch polysaccharide;
wherein the first, second, and third materials remain in solution for all pH values of the solution within a selected range of pH values.

4. A dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and, combinations thereof;
(b) a second material selected from the group consisting of an aqueous soluble starch conversion product, a resistant maltodextrin, an aqueous soluble non-starch polysaccharide, and combinations thereof; and,
(c) a third material selected from aqueous soluble ginseng extract, aqueous soluble red ginseng extract, and combinations thereof;
wherein the first, second materials, and third material remain in solution for all pH values of the solution within a selected range of pH values.

5. A dried form of a primary aqueous solubilized bile acid formulation comprising:
(a) a first material selected from the group consisting of a bile acid, an aqueous soluble derivative of a bile acid, a bile acid salt, a bile acid conjugated with an amine by an amide linkage, and combinations thereof; and
(b) a second material selected from the group consisting of an aqueous soluble starch conversion product, a non-starch polysaccharide, and combinations thereof,
wherein the first and second materials both remain in solution for all pH values of the solution within a selected range of pH values.

6. The dried form of a primary aqueous solubilized bile acid formulation of claim 5 further comprising riluzole, wherein the solid form is an oral solid dosage form.

7. The dried form of a primary aqueous solubilized bile acid formulation of claim 5, wherein in the primary aqueous solubilized bile acid formulation further comprises suspended insoluble bismuth compound and wherein the solid form is an oral solid dosage form.

8. The dried form of a primary aqueous solubilized bile acid formulation of claim 5, wherein the bile acid is selected from the group consisting of ursodeoxycholic acid, chenodeoxycholic acid, cholic acid, hyodeoxycholic acid, deoxycholic acid, 7-oxolithocholic acid, lithocholic acid, iododeoxycholic acid, iocholic acid, tauroursodeoxycholic acid, taurochenodeoxycholic acid, taurodeoxycholic acid, glycourso-deoxycholic acid, taurocholic acid, glycocholic acid, their derivatives at a hydroxyl or carboxylic acid group on the steroid nucleus, their salts, or their conjugates with amines.

9. The dried form of a primary aqueous solubilized bile acid formulation of claim 5, wherein the aqueous soluble starch conversion product is selected from the hydrolyzed starch having Dextrose Equivalence (DE) ranged from 4 to 40 such as Maltrine® M040 (DE=5, maltodextrin), Maltrin® M050 (DE=5, maltodextrin), Maltrine® M100 (DE=10, maltodextrin), Maltrin® M150 (DE=15, maltodextrin), Maltrine® M180 (DE=18, maltodextrin), Maltrin® M200 (DE=20, corn syrup solids), and Maltrin® M250 (DE=25, corn syrup solids).

10. The dried form of a primary aqueous solubilized bile acid formulation of claim 1, wherein in the primary aqueous solubilized bile acid formulation further comprises a dissolving agent.

11. The dried form of a primary aqueous solubilized bile acid formulation of claim 10, wherein the dissolving agent is maltodextrin.

12. The dried form of a primary aqueous solubilized bile acid formulation of claim 1, wherein in the primary aqueous solubilized bile acid formulation further comprises a branched chain amino acid selected from the group consisting of leucine, isoleucine, valine, and mixtures thereof.

13. The dried form of a primary aqueous solubilized bile acid formulation of claim 5, wherein the primary aqueous solubilized bile acid formulation further comprises an aqueous soluble reaction product between a bismuth ion and a chelator.

14. The dried form of a primary aqueous solubilized bile acid formulation of claim 13, wherein the chelator is selected from the group consisting of citric acid, tartaric acid, malic acid, lactic acid, eidetic acid and alkalies, and combinations thereof.

15. The dried form of a primary aqueous solubilized bile acid formulation of claim 13, wherein the bismuth compound is selected from the group consisting of bismuth citrate, bismuth sulfate, and bismuth subnitrate.

16. The dried form of a primary aqueous solubilized bile acid formulation of claim 13, wherein the bismuth compound is selected from the group consisting of bismuth subcarbonate, bismuth subgallate or bismuth subsalicylate.

17. The dried form of a primary aqueous solubilized bile acid formulation of claim 5, wherein the primary aqueous solubilized bile acid formulation further comprises one or more additional bile acids, aqueous soluble derivatives of bile acid, bile acid salts, and amine-conjugated bile acids conjugated by an amide linkage.

18. The dried form of a primary aqueous solubilized bile acid formulation of claim 5, wherein the second material is an aqueous soluble non-starch polysaccharide is selected from the group consisting of guar gum, pectin, cellulose, glycogen, and inulin.

19. The dried form of a primary aqueous solubilized bile acid formulation of claim 5, wherein the primary aqueous solubilized bile acid formulation further comprises a disintegrant.

20. The dried form of a primary aqueous solubilized bile acid formulation of claim 19, wherein the disintegrant is selected from the group consisting of Veegum HV, methylcellulose, agar, bentonite, natural sponge, cation exchange resins, alginic acid, guar gum, citrus pulp, and carboxymethylcellulose, clays, celluloses, aligns, gums, and cross-linked polymers (crospovidone), cross-linked cellulose (Croscarmelose), and cross-linked starch (sodium starch glycolate).

21. The dried form of a primary aqueous solubilized bile acid formulation of claim 5, wherein the dried form comprises film-coated granules, said film comprising a polymer and a plasticizer.

22. The dried form of a primary aqueous solubilized bile acid formulation of claim 21, wherein the polymer is selected from the group consisting of hydroxylpropyl methylcellulose ether, methylcellulose ether, methacrylate copolymer and methyl methacrylate copolymer.

23. The dried form of a primary aqueous solubilized bile acid formulation of claim 21, wherein the plasticizer is selected from the group consisting of glycerin, propylene glycol, polyethylene glycol, triacetin, acetylated monoglyceride, triethyl citrate, and dithyl phthalate.

24. The dried form of a primary aqueous solubilized bile acid formulation of claim 21, wherein the polymer is an enteric polymer selected from the group consisting of cellulose acetate phthalate (CAP), which is capable of functioning effectively as an enteric coating at pH greater than 6, polyvinyl acetate phthalate (PVAP), methacrylic acid-methacylic acid ester copolymers, cellulose acetate trimellitate (CAT), carboxymethyl ethylcellulose (CMEC), and hydroxylpropyl methylcellulose acetate succinate (HPMCAS).

25. The dried form of a primary aqueous solubilized bile acid formulation of claim 5, wherein the aqueous solubilized bile acid formulation further comprises at least one pharmaceutical in a pharmaceutically effective amount.

26. The dried form of a primary aqueous solubilized bile acid formulation of claim 25, wherein the pharmaceutical compound is selected from the group consisting of octreotide, sildenafil citrate, calcitriol, dihydrotachysterol, ampomorphine, yohimbin, trazodone, acyclovir, cidofovir, delavirdine-mesylate, didanosine, famciclovir, forscarnet sodium, fluorouracil, ganciclovir sodium, idoxuridine, interferon-α, interferon-β, interferon-γ, lamivudine, nevirapine, penciclovir, ribavirin, stavudine, trifluridine, valacyclovir.HCl, zalcitabine, zidovudine, indinavir.H₂SO₄, ritonavir, nelfinavir.CH₃SO₃H, saquinavir.CH₃SO₃H, d-penicillamine, chloroquine, hydroxychloroquine, aurothioglucose, gold sodium thiomalate, auranofin levaminsole, DTC, isoprinosine, methyl inosine monophosphate, muramyl dipeptide, diazoxide, hydralazine.HCl, minoxidil, dipyridamole, isoxsuprine.HCl, niacin, nylidrin.HCl, phentolamine, doxazosin.CH₃SO₃H, prazosin.HCl, terazocin.HCl, clonidine.HCl, nifedipine, molsidonine, amiodarone, acetylsalicylic acid, verapamil, diltiazem, nisoldipine, isradipine, bepridil, isosorbide.dinitrate, pentaerythrytol.tetranitrate, nitroglycerin, cimetidine, famotidine, nizatidine, ranitidine, lansoprazole, omeprazole, misoprostol, sucralfate, metoclopramide.HCl, erythromycin, alprostadil, albuterol, pirbuterol, terbutaline.H₂SO₄, salmetrol, aminophylline, dyphylline, ephedrine, ethylnorepinephrine, isoetharine, isoproterenol, metaproterenol, n.docromil, oxy triphylline, theophylline, bitolterol, fenoterol, budesonide, flunisolide, beclomethasone.dipropionate, fluticasone.propionate, codeine, codeine sulfate, codeine phosphate, dextromethorphan.HBr, triamcinolone.acetonide, montelukast sodium, zafirlukast, zileution, cromolyn sodium, ipratropium bromide, nedocromil sodium benzonate, diphenhydramine.HCl, hydrocodone.bitartarate, methadone.HCl, morphine sulfate, acetylcysteine, guaifenesin, ammonium carbonate, ammonium chloride, antimony potassium tartarate, glycerin, terpin.hydrate, colfosceril palmitate, atorvastatin.calcium, cervastatin.sodium, fluvastatin.sodium, lovastatin, pravastatin.sodium, simvastatin, picrorrhazia kurrva, andrographis paniculata, moringa oleifera, albizzia lebeck, adhata vasica, curcuma longa, momordica charantia, gymnema sylvestre, terminalia arjuna, azadirachta indica, tinosporia cordifolia, metronidazole, amphotericin B, clotrimazole, fluconazole, haloprogin, ketoconazole, griseofulvin, itraconazole, terbinafin.HCl, econazole.HNO₃, miconazole, nystatin, oxiconazole.HNO₃, sulconazole.HNO₃, cetirizine.2HCl, dexamethasone, hydrocortisone, prednisolone, cortisone, catechin and its derivatives, glycyrrhizin, glycyrrhizic acid, betamethasone, ludrocortisone.acetate, flunisolide, fluticasone.propionate, methyl prednisolone, somastostatin, lispro, glucagon, acarbose, chlorpropamide, glipizide, glyburide, metformin.HCl, repaglinide, tolbutamide, colchicine, sulfinpyrazone, allopurinol, piroxicam, tolmetin sodium, indomethacin, ibuprofen, diflunisal, mefenamic acid, naproxen, trientine, sulindac, sulindac sulfone, selenium compounds insuline, heparin, ampicillin, amantadine, rimantadine, proinsulin, celecoxib, budesonide, salicylic acid and its derivatives. Vitamin E, vitamin C, superoxide dismutase (SOD), N-acetylcysteine, 21-aminosteroid such as lazaroids, U74389F and U74006F, catalase (CAT), putrescine-modified catalase (PUT-CAT), estrogen, alpha-lipoic acid, selegiline, desferrioxanmine, d,1-penicillamine, alpha and beta-carotene, retinol, selenium, gingko biloba, riluzole, flupirtine, pifithrin-alpha, CGP 3466B/TCH346, CPI-1189, CEP-1347, and coenzyme Q 10.

27. The dried form of a primary aqueous solubilized bile acid formulation of claim 5 further comprising an additive.

28. The dried forms of a primary aqueous solubilized bile acid formulation of claim 27, wherein the additive is selected from the group consisting of a diluent, a lubricant, a binder, a filler, and combinations thereof.

29. A method of preparing a dried form of a primary aqueous solubilized bile acid formulation comprising:
preparing an primary aqueous solubilized bile acid formulation comprising:
a first material selected from the group consisting of a bile acid, an aqueous soluble derivative of a bile acid, a bile acid salt, a bile acid conjugated with an amine by an amide linkage, and combinations thereof; and
a second material selected from the group consisting of an aqueous soluble starch conversion product, a non-starch polysaccharide, and combinations thereof,
wherein the first and second materials both remain in solution for all pH values of the solution within a selected range of pH values.
removing water from the primary aqueous solubilized bile acid formulation by a granulation method selected from the group consisting of wet granulation, fluid-bed granulation, dry granulation, spheronization, spray-drying, evaporation, lyophilization, and combinations thereof,
wherein a dry form is produced.

30. The method of claim 29 further comprising sonicating the primary aqueous solubilized bile acid formulation.

31. The method of claim 29, wherein the dry form comprises granules.

32. The method of claim 31 further comprising coating a granule having an enteric polymer.

33. The method of claim 32, wherein the enteric polymer is selected from the group consisting of cellulose acetate phthalate (CAP), polyvinyl acetate phthalate (PVAP), methacrylic acid-methacylic acid ester copolymers, cellulose acetate trimellitate (CAT), carboxymethyl ethylcellulose (CMEC), and hydroxylpropyl methylcellulose acetate succinate (HPMCAS).

34. The method of claim 29 further comprising forming film comprising a polymer and a plasticizer on the dry form.

35. The method of claim 34, wherein the polymer is selected from the group consisting of hydroxylpropyl methylcellulose ether, methylcellulose ether, methacrylate copolymer and methyl methacrylate copolymer.

36. The method of claim 34, wherein the plasticizer is selected from the group consisting of glycerin, propylene glycol, polyethylene glycol, triacetin, acetylated monoglyceride, and triethyl citrate and dithyl phthalate.

37. The method of claim 29, wherein the removal of water is by spheronization and spherical pellets are formed.

38. The method of claim 29, wherein the primary aqueous solubilized bile acid formulation further comprises sodium bicarbonate and an acidulant.

39. The method of claim 38, wherein the amount of sodium bicarbonate is about ten times the amount of the first material by weight.

40. The method of claim 38, wherein the primary aqueous solubilized bile acid formulation comprises about twenty percent more acidulant than sodium bicarbonate by weight.

41. The method of claim 38, wherein the acidulant is selected from the group consisting of tartaric acid and citric acid.

42. The method of claim 29, further comprising completely dissolving the solid form in water in a neutral or slightly acidic reaction.
